(19) United States
(12) Patent Application Publication
(10) Pub. No.: US 2010/0114150 A1
(43) Pub. Date: May 6, 2010

(54) DUODENAL STIMULATION DEVICES AND METHODS FOR THE TREATMENT OF CONDITIONS RELATING TO EATING DISORDERS

(75) Inventor: Elad Magal, Ramat-HaSharon (IL)

Correspondence Address: HESLIN ROTHENBERG FARLEY & MESITI PC, 5 COLUMBIA CIRCLE, ALBANY, NY, 12203, US

(73) Assignee: DUOCURE, INC., Ramat-HaSharon (IL)

(21) Appl. No.: 12/525,855

(22) PCT Filed: Feb. 7, 2008
(86) PCT No.: PCT/IL08/00169

§ 371 Date: Dec. 21, 2009

Related U.S. Application Data
(60) Provisional application No. 60/899,890, filed on Feb. 7, 2007.
(60) Provisional application No. 60/929,385, filed on Jun. 25, 2007.

(51) Int. Cl. A61M 29/02

(52) U.S. Cl. 606/192

(57) ABSTRACT

A device useful for treatment of conditions relating to an eating disorder comprises: a) a duodenum obstructing component configured to partially obstruct the lumen of a duodenum in which deployed, preferably so as to reduce the rate of passage of materials through the duodenum; and b) an anchoring component configured to substantially maintain a position of the obstructing component inside a duodenum wherein deployed. The obstructing component does not block entry of food into the duodenum but rather causes a given volume of food that enters the duodenum to induce a greater degree of satiety and/or to induce a perception of satiety for a longer period of time and/or to induce a perception of satiety faster than otherwise.

### CROSS REFERENCE TO RELATED APPLICATIONS

This application is a national stage filing under section 371 of International Application No. PCT/IL2008/000169, filed on Feb. 7, 2008, and published in English on Aug. 14, 2008 as WO 2008/096362 and claims priority of U.S. application No. 60/899,890 filed on Feb. 7, 2007, and U.S. application No. 60/929,385 filed on Jun. 25, 2007, the entire disclosure of these applications being hereby incorporated herein by reference.

### RELATED APPLICATIONS

The present invention claims priority from U.S. Provisional Patent Application No. 60/899,890 filed on 7 Feb. 2007 which is included by reference as if fully set forth herein.

### FIELD AND BACKGROUND OF THE INVENTION

The present invention relates to the treatment of conditions related to eating disorders such as obesity and overeating. Specifically the invention relates to methods and devices that, by partially obstructing the lumen of the duodenum, provide a beneficial effect for treating conditions related to eating disorders, in embodiments, for example, by prolonging, intensifying or accelerating the onset of the perception of satiety induced by consumption of a given volume of food.

Embodiments of the present invention are related to and in embodiments are complementary to the teachings of the PCT patent application published as WO2006/035446 of the Applicant which is included by reference as if fully set forth herein and to the teachings of U.S. patent application No. 60/903,289 of the Inventor which is included by reference as if fully set forth herein.

Obesity is a result, a symptom and/or a cause of many pathological conditions. An accepted way to reduce the degree of obesity is by reducing caloric intake of an obese subject over an extended period of time.

It is known to treat obesity by dieting, that is, an obese person reduces caloric intake to below the amount expended. As a result, bodily tissue is consumed to provide energy, leading to a loss of weight. Dieting requires a very high level of personal discipline and motivation over a long period of time which is known to be difficult, especially when a person is continuously exposed to readily available high-caloric food and to other hunger-inducing stimuli.

It is known that the desire to eat is, in a large part, driven by hunger which dissipates upon the perception of satiety. As a result of eating, gastric mechanoreceptors detect the degree of distension of the stomach and release satiety factors. Known satiety factors that are released to control food ingestion include Cholecystokinin (CCK), Bombesin, Gastrin-releasing peptide (GRP), Glucagon, Glucagon-like peptide (GLP-1), Enterostatin and Ghrelin. As there is a delay between the time of food ingestion and the release of the satiety factors, it is common for a person to overeat. Apart from the direct weight gain caused by consuming too much food, overeating also causes the base volume of the stomach to increase and the gastric mechanoreceptors to become insensitive to small increases of stomach volume. Thus, a positive-feedback loop with negative consequences is generated where a person inherently overeats as satiety is perceived only after satiety is reached, so that the person overeats, reducing the sensitivity of the satiety sensors, so that the indication of satiety is delayed even further.

A concept that has been considered for the treatment of obesity is to increase the magnitude of satiety perceived by the consumption of a relatively small volume of food, the idea being that a person will feel satiated after having eaten an amount of food that is low enough to lead to a loss of weight. Various interventional methods have been contemplated for increasing the magnitude of satiety perceived by consumption of a relatively small amount of food.

Lap bands (e.g., such as available from Allergan Inc., Irvine, Calif., USA) are used to constrict a stomach in the proximity of the esophagus thus defining a small gastric pouch that functions as a reduced size stomach. Even a relatively small volume of food consumed causes the pouch to stretch, stimulating gastric mechanoreceptors to induce a perception of satiety. Lap bands may lead to side effects such as reflux and nausea and limit the type of foods that a person can eat. There have been reports of lap bands migrating through the gastric walls to enter the gastric cavity.

Intragastric balloons (e.g., BioEnterics® Intragastric Balloon System available from Inamed Health, a division of Allergan, Santa Barbara, Calif., USA) are deployed in the stomach to occupy a significant proportion of the gastric volume. The volume of the balloon together with a relatively small volume of food consumed stretches the gastric wall to the extent that gastric mechanoreceptors are stimulated to induce a perception of satiety. It has been found that the elasticity of the stomach is such that over an extended period of time, the stomach and the gastric mechanoreceptors become insensitive to the presence of the intragastric balloon which ultimately becomes ineffective.

An alternative approach for treating obesity and other related conditions is taught in PCT patent application PCT/IL2005/001053 published as WO 2006/035446 of the Applicant. A central concept taught therein is the delivery of a beneficial stimulus at the “right time” (only when needed) at the “right place” (to a localized part of the body where most effective) which allows the “right dose” to be administered (no over- or under-dosing). Specifically, WO 2006/035446 teaches a device capable of sensing a physiological change associated with food ingestion or hunger (i.e., identifying the “right time”) and a mechanism adapted for directly stimulating a region of the body responsive to a gastrointestinal satiety agent which is implanted in the body (i.e., the “right place”). Specific mechanisms for sensing a physiological change associated with food ingestion or hunger include muscle activity sensors and pressure sensors. Specific mechanisms adapted for stimulation include drug dispensers, space-filling balloons, vagal-mechanoreceptor stimulating balloons and nerve-stimulating electrodes. Upon detection of a physiological change associated with food ingestion or hunger, the stimulation mechanism is activated inducing a perception of satiety. By detecting that a person is about to eat or has started eating before having consumed too much food and then stimulating a perception of satiety, the teachings of WO 2006/035446 treat or control over-eating and related disorders such as obesity.

Further, in WO 2006/035446 is taught the induction of the perception satiety by administration of an active agent (such as an active pharmaceutical ingredient) specifically to a region where that active agent is preferentially active, for example, the duodenum, the antral sphincter and the gastrointestinal wall where chemoreceptors sensitive to the active agent are found.

In U.S. patent application No. 60/903,289 of the Inventor is taught a method of administering an active agent by spraying a composition including the active agent directly at a luminal wall of the gastrointestinal tract. Therein is demonstrated that a composition including an analogue of a peptide hormone that is a satiety agent (CCK8, an analogue of CCK) can be administered and interacts with chemoreceptors on the luminal wall of the duodenum to induce a perception of satiety.

It would be highly advantageous to have methods and/or devices that induce a perception of satiety more quickly and/or for a longer time and/or that is more intense for a given volume of food consumed than otherwise and that have advantages over the methods known in the art.

### SUMMARY OF THE INVENTION

Embodiments of the present invention successfully address at least some of the shortcomings of the prior art by providing a method and a device for inducing an increased a perception of satiety by partially obstructing the lumen of the duodenum. In embodiments, the partial obstruction reduces the rate of passage of materials through the duodenum, so that for a given volume of food, duodenal mechanoreceptors are stimulated more intensely and/or more quickly and/or for a longer period of time than without the obstruction. In embodiments, such stimulation leads to a beneficial effect, for example, a perception of satiety.

Thus, according to an aspect of some embodiments of the present invention there is provided a method of treatment of a condition related to an eating disorder, comprising: partially obstructing the lumen of a duodenum of a subject (whether a human or a non-human animal) suffering from the condition so as to substantially reduce the rate of passage of materials therethrough, leading to an effect beneficial for treating the condition.

In embodiments, the partial obstructing comprises: providing a device including a duodenum obstructing component; and deploying the device so that the obstructing component is deployed inside the lumen of the duodenum. In embodiments, the deploying of the obstructing component is such that the partial obstruction substantially commences no more than about 5 cm from a pyloric sphincter associated with the duodenum. In embodiments, the deploying of the obstructing component is such that the partial obstruction commences in the superior portion of the duodenum.

In embodiments, the obstructing component is flexible or otherwise deformable. In embodiments, the obstructing component comprises at least one coiled section, in embodiments at least one coiled section comprising a conical coil shape. In embodiments the coiled section is flexible. In embodiments, the coiled section is configured to axially stretch. In embodiments, the coiled section is configured to axially bend.

In embodiments, the coiled section comprises a coiled tube. In embodiments, the obstructing component comprises an inflatable balloon including an internal volume.

In embodiments, the method further comprises: anchoring the obstructing component in place in the duodenum.

In embodiments, the method further comprises holding the obstructing component in place at least in part by a component that passes through the pyloric sphincter into the duodenum from the stomach associated with the duodenum.

In embodiments, the method further comprises varying the degree of the partial obstruction when necessary. In embodiments, the necessity of varying the degree of partial obstruction is a result of an evaluation of the degree of the beneficial effect. In embodiments, the necessity of varying the degree of the partial obstruction is determined by detection of an event of significance for varying the degree of the partial obstruction. For example, in embodiments, an event of significance for varying the degree of the partial obstruction is a physiological change indicative of, for example food ingestion or hunger, such as increased gastrointestinal tract activity. In embodiments, the varying of the degree of the partial obstruction is initiated a specified time subsequent to detection of the event. In embodiments, the detection of the event is automatically performed with the help of an event detector. In embodiments the detection of the event is manual, for example is performed by a care-giver, medical professional or the subject self. In embodiments varying the degree of partial obstruction of the lumen of the duodenum comprises changing a conformation of an obstructing component. For example, in embodiments an obstructing component is a balloon and the degree of partial obstruction is varied by changing the degree of inflation of the balloon. For example, in embodiments, an obstructing component comprises a coiled section and the degree of partial obstruction is varied by changing the radius of some or all of the loops making up the coiled section. For example, in embodiments, an obstruction is a substantially conical coiled section and the degree of partial obstruction is varied by changing the length of the coil so as to change the size of the gaps between the loops making up the coil.

In embodiments, the method of the present invention comprises a stimulation of the duodenum in addition to the stimulation caused by the partial obstruction. For example, in embodiments, the method of the present invention further comprises applying pressure to at least a portion of a luminal wall of the duodenum so as to stimulate duodenal mechanoreceptors, for example to induce a perception of satiety.

In embodiments, the method of the present invention further comprises, when necessary, applying at least one additional stimulus to the duodenum. In embodiments, the necessity of the application of an additional stimulus is periodic. In embodiments, the necessity of the application of an additional stimulus is determined by detection of an event of significance for application of the additional stimulus. For example, in embodiments, an event of significance for applying an additional stimulus is a physiological change indicative of, for example food ingestion or hunger, such as increased gastrointestinal tract activity. In embodiments, the application of an additional stimulus is initiated a specified time subsequent to detection of the event. In embodiments, the detection of the event is automatically performed with the help of an event detector. In embodiments the detection of the event is manual, for example is performed by a care-giver, medical professional or the subject self.

In embodiments, the additional stimulus comprises applying pressure for a period of time (generally no greater than about 60 minutes) to at least a portion of a luminal wall of the duodenum, preferably so that the applied pressure stimulates mechanoreceptors so as to induce a perception of satiety. In embodiments, during the period of time the pressure is substantially constant. In embodiments, during the period of time the pressure is pulsatile.

In embodiments, the additional stimulus comprises electrically stimulating the duodenum for a period of time, generally for a period of time no greater than about 60 minutes, preferably so that the electrical stimulation induces a perception of satiety. In embodiments the electrical stimulation is of nerves in the duodenum such as the vagus nerves.

In embodiments, the additional stimulus comprises a dose of a pharmaceutical composition comprising an active agent and a pharmaceutically acceptable carrier administered in the duodenum, preferably so that the administration of the pharmaceutical composition induces a perception of satiety.

According to an aspect of some embodiments of the present invention there is also provided a device for treatment of a condition related to an eating disorder, comprising: a) a duodenum obstructing component configured to partially obstruct the lumen of a duodenum in which deployed, in embodiments so as to substantially reduce the rate of passage of materials through the duodenum; and b) an anchoring component configured to substantially maintain a position of the obstructing component inside the lumen of a duodenum wherein deployed.

In embodiments, the anchoring component is substantially tensionless anchoring. In embodiments, the anchoring component is configured to substantially maintain the proximal end of the obstructing component no more than about 5 cm from a pyloric sphincter associated with the duodenum. In embodiments of a device of the present invention, the anchoring component is configured to substantially maintain the obstructing component in the superior portion of a duodenum in which deployed.

In embodiments, the anchoring component is configured to penetrate into gastrointestinal tissue so as to maintain the obstructing component in the desired location in a duodenum. In embodiments, the anchoring component is configured to apply outwards pressure to luminal walls of a gastrointestinal tract so as to maintain the obstructing component in the desired location in a duodenum. In embodiments, at least part of the anchoring component is configured to pass through a pyloric sphincter from a stomach associated with the duodenum.

In embodiments, the obstructing component is configured to have at least two conformations, a first conformation and a second conformation providing different degrees of partial duodenal obstruction.

In embodiments, a device of the present invention further comprises a conformation-determining component configured to transform the obstructing component, preferably reversibly, from the first conformation to the second conformation while the obstructing component is deployed within a duodenum. For example, in embodiments, the conformation-determining component includes an internal volume and the transformation is effected by a process including transport of fluid into and/or out of the internal volume. For example, in embodiments, the conformation-determining component comprises a heat-sensitive element and the transformation is effected by a process including changing the temperature of the heat-sensitive element.

In embodiments, the conformation-determining component is configured for manual manipulation.

In embodiments, a device of the present invention further comprises an actuator configured to trigger the conformation-determining component to transform the obstructing component from the first conformation to the second conformation.

In embodiments, a device of the present invention further comprises a timer functionally associated with the actuator. In embodiments, the timer is configured to activate the actuator to trigger the conformation-determining component for a specified period of time. In embodiments, the timer is configured to periodically activate the actuator to trigger the conformation-determining component. In embodiments, a device of the present invention further comprises an event detector functionally associated with the actuator, so that the actuator triggers the conformation-determining component as a consequence of detection of an event of significance for changing the conformation. In embodiments, the event detector comprises an electrode configured for deployment in a body. In embodiments, the event detector is configured to detect a physiological change (e.g., increased gastrointestinal tract activity) indicative of a member of the group consisting of food ingestion and hunger. In embodiments, a device of the present invention further comprises a timer functionally associated with the actuator and with the event detector. In embodiments, the timer is configured to activate the actuator to trigger the conformation-determining component a specified period of time subsequent to detection of an event by the event detector.

In embodiments, a device of the present invention further comprises a component configured to apply a stimulus to a duodenum in which deployed in addition to the stimulus caused by the partial obstruction of a duodenum by the obstructing component. In embodiments, the component configured to apply an additional stimulus is substantially the obstructing component. In embodiments, the component configured to apply an additional stimulus is distinct from the obstructing component.

In embodiments, the component is configured to apply the additional stimulus substantially continuously.

In embodiments, a device of the present invention further comprises an actuator configured to trigger the additional stimulus applying component to apply an additional stimulus to the duodenum. In embodiments, a device of the present invention further comprises a timer functionally associated with the actuator. In embodiments, the timer is configured to activate the actuator to trigger the additional stimulus applying component for a specified period of time. In embodiments, the timer is configured to periodically activate the actuator to trigger the additional stimulus applying component. In embodiments, a device of the present invention further comprises an event detector functionally associated with the actuator, so that the actuator triggers the additional stimulus applying component as a consequence of detection of an event of significance for applying the additional stimulus. In embodiments, the event detector comprises an electrode configured for deployment in a body. In embodiments, the event detector is configured to detect a physiological change (e.g., increased gastrointestinal tract activity) indicative of a member of the group consisting of food ingestion and hunger. In embodiments, a device of the present invention further comprises a timer functionally associated with the actuator and with the event detector. In embodiments, the timer is configured to activate the actuator to trigger the additional stimulus applying component a specified period of time subsequent to detection of an event by the event detector.

In embodiments, a device of the present invention further comprises a mechanoreceptor-stimulating component as an additional duodenum stimulating component, the mechanoreceptor-stimulating component configured to apply an outwards pressure to at least a portion of a luminal wall of a duodenum in which deployed. In embodiments, the mechanoreceptor-stimulating component is configured to have at least two conformations, a first conformation and a second conformation providing different degrees of the outwards pressure. In embodiments, a surface of the mechanoreceptor-stimulating component configured to contact the portion of the luminal wall includes duodenal wall stimulating protuberances such as grooves, strips, studs or spikes. Typically, such protuberances are between about 50 micrometers and 3 millimeters in height.

In embodiments, a device of the present invention further comprises a mechanoreceptor-stimulating component conformation determiner configured to transform, preferably reversibly, the mechanoreceptor-stimulating component from the first conformation to the second conformation while the mechanoreceptor-stimulating component is deployed within a duodenum. For example, in embodiments, the mechanoreceptor-stimulating component conformation determiner includes an internal volume and the transformation is effected by a process including transport of fluid into and/or out of the internal volume. For example, in embodiments, the mechanoreceptor-stimulating component conformation determiner comprises a heat-sensitive element and the transformation is effected by a process including changing the temperature of the heat-sensitive element.

In embodiments, a device of the present invention further comprises an electrical stimulator component as an additional duodenum stimulating component configured to electrically stimulate at least a portion of a luminal wall of a duodenum in which deployed. In embodiments, the electrical stimulator is configured to stimulate nerves in a duodenum in which the device is deployed, such as the vagus nerves in order to induce a perception of satiety.

In embodiments, a device of the present invention further comprises an active agent dispensing component configured to administer a dose of a pharmaceutical composition comprising an active agent and a pharmaceutically acceptable carrier. In embodiments, the active agent dispensing component comprises a sprayer configured to direct a spray towards a luminal wall of a duodenum in which deployed. In embodiments, a device of the present invention further comprises an active agent reservoir functionally associated with the active agent dispensing component and also a pressure generator configured to transport a composition held in the active agent reservoir to be dispensed through the active agent dispensing component.

In embodiments of a device of the present invention, an obstructing component comprises at least one coiled section. In embodiments, the cross section of the elongate element is, for example, round or square. In embodiments, for example, the cross section of the elongate element has a greater and a lesser dimension, e.g. is oval or rectangular.

In embodiments, the length of at least one such coiled section is at least about 1 cm. In embodiments, the length of at least one such coiled section is no more than about 5 cm. In embodiments, the distance between any two loops of such a coiled section is at least about 0.5 cm. In embodiments, at least one such coiled section comprises a conical coil shape.

In embodiments, at least one such coiled section comprises a solid cross section, e.g., such as a wire or the like.

In embodiments, at least one such coiled section comprises a coiled tube including an axial channel passing therethrough. In embodiments, the coiled section comprises an open tube with an open axial channel running therethrough, for example to allow deployment of the coiled section in a duodenum with the help of a delivery guide wire that straightens the coiled section.

In embodiments, at least one such coiled section comprises a closed tube with an elongated axial internal volume inside the coiled section. In embodiments, a device of the present invention further comprises a pressure generator configured to force fluid into and/or out of the internal volume, so as to change a conformation of the coiled section. In embodiments, a device of the present invention further comprises a fluid reservoir functionally associated with the pressure generator. In embodiments, such a fluid reservoir is configured for implantation in a body, e.g., in the gastrointestinal tract, the stomach or subcutaneous implantation.

In embodiments, a coiled section comprises a heat sensitive element configured to change conformation upon changing of temperature, e.g., to a larger or smaller radius coil or to a shorter or longer coil where the loops making up the coil are closer or further apart. In embodiments, a device of the present invention further comprises a heating element functionally associated with the heat-sensitive element so as to function as a conformation determining component by heating the heat sensitive element. In embodiments, a device of the present invention further comprises a power supply configured to supply power to the heating element

In embodiments of a device of the present invention, an obstructing component comprises an inflatable balloon including an internal volume. In embodiments, a device of the present invention further comprises a pressure generator configured to force fluid into and/or out of the internal volume of the balloon, so as to change a conformation of the balloon. In embodiments, a device of the present invention further comprises a fluid reservoir functionally associated with the pressure generator, so that the pressure generator is configured to transport fluid from the fluid reservoir into the internal volume of the balloon and/or from the internal volume of the balloon into the fluid reservoir. In embodiments, such a fluid reservoir is configured for implantation in a body, e.g., in the gastrointestinal tract, the stomach or subcutaneous implantation.

In embodiments, an inflatable balloon is elongated and configured to have a substantially straight conformation.

In embodiments, the inflatable balloon is elongated and configured to have a coiled conformation when partially inflated. In such embodiments, the diameter of the balloon typically has a cross section (in the inflated conformation) of less than about 1 cm and even less than about 0.5 cm.

In embodiments, an inflatable balloon is annular with a balloon lumen and an external diameter. In embodiments, such an inflatable balloon is configured to have a smaller diameter balloon lumen with increased inflation. In embodiments, such an inflatable balloon is configured to have a greater external diameter with increased inflation.

According to an aspect of some embodiments of the present invention there is also provided a method of treatment of a condition related to an eating disorder, comprising: a) providing a device of the present substantially as described above; b) deploying an obstructing component of the device in the lumen of a duodenum of a subject suffering from the condition so as to partially obstruct the lumen of the duodenum; and c) using an anchoring component of the device to substantially maintain a position of the obstructing component inside the duodenum; thereby reducing the rate of passage of materials through the duodenum, leading to an effect beneficial for treating the condition.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Herein, the term “active agent” is meant to include chemical, biological or pharmaceutical materials including any natural or synthetic chemical or biological substance that influences a cell, an organ or organism to which the material is administered. Typical active agents include but are not limited to active pharmaceutical ingredients, antibodies, antigens, biological materials, chemical materials, chemotherapeutic agents, diagnostic agents, DNA, drugs, dyes, enzymes, foodstuffs, hormones, immunogens, ligands, liposomes, markers, nanoparticles, nucleic acids, nutrients, physiological media, proteins, radio-labeled markers, RNA, selective toxins, therapeutic monoclonal antibodies, toxins and vaccines and especially peptides and peptide hormones.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the some embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 depicts an embodiment of a device of the present invention including a conical coiled duodenum obstructing component deployed in the superior portion of a duodenum;

FIGS. 2A and 2B depict an embodiment of a device of the present invention including a conical coiled duodenum obstructing component and a pharmaceutical composition sprayer deployed in the superior portion of a duodenum;

FIGS. 3A, 3B and 3C depict an embodiment of a device of the present invention comprising a heat-sensitive obstructing component having a reversibly variable conformation allowing variation of the degree of obstruction of a duodenum in which deployed;

FIGS. 4A, 4B and 4C depict an embodiment of a device of the present invention comprising an obstructing component having a reversibly variable conformation allowing variation of the degree of obstruction of a duodenum in which deployed, the variation of conformation effected by the transport of fluid into and out of a chamber;

FIGS. 5A and 5B depict an embodiment of a device of the present invention comprising an obstructing component that is substantially a sausage-shaped balloon deployed in the superior portion of a duodenum;

FIG. 6 depicts an embodiment of a device of the present invention comprising an obstructing component that is substantially an annular balloon deployed in the superior portion of a duodenum;

FIG. 7 depicts an embodiment of a device of the present invention comprising an obstructing component that is substantially an annular balloon deployed in the superior portion of a duodenum, the balloon including a sleeve configured to limit the extent of outward expansion of the balloon;

FIGS. 8A, 8B and 8C depict an embodiment of a device of the present invention comprising an obstructing component in the shape of a conical coil having a reversibly variable conformation allowing variation of the degree of obstruction of a duodenum in which deployed by changing the length of the obstructing component;

FIGS. 9A, 9B and 9C depict an embodiment of a device of the present invention including a coiled duodenum obstructing component and an inflatable balloon as an additional duodenum obstructing component, both obstructing components having a reversibly variable conformation allowing variation of the degree of obstruction of a duodenum in which deployed, a pharmaceutical composition sprayer, stimulation electrodes for directly electrically stimulating the walls of a duodenum in which deployed and detection electrodes to detect events of significance for applying stimuli using the device, deployed in the superior portion of the duodenum;

FIGS. 10A and 10B depict an embodiment of a device of the present invention including a coiled duodenum obstructing component and an inflatable balloon as an additional duodenum obstructing component, the balloon having a reversibly variable conformation allowing variation of the degree of obstruction of a duodenum in which deployed, a pharmaceutical composition sprayer and detection electrodes to detect events of significance for applying stimuli using the device, deployed in the superior portion of the duodenum; and

FIGS. 11A-11F depict an embodiment of a device of the present invention including a composition feeder tube that passes from outside the body of a subject, into the stomach cavity, through the pyloric sphincter to a combined sprayer/obstructing component deployed in the duodenum.

### DESCRIPTION OF EMBODIMENTS

The present invention relates to the treatment of conditions relating to eating disorders such as obesity and overeating. Specifically, embodiments of the invention relate to methods and devices acting upon the duodenum to induce a perception of satiety with a relatively small volume of food.

The duodenum, the most proximal part of the small intestine, is approximately 24 cm long. In an adult the course of the duodenum describes an almost 270° imperfect circle divided into four roughly linear portions: the first (superior) portion; the second (descending) portion; the third (transverse) portion; and the fourth (ascending) portion.

The superior portion of the duodenum, is about 5 cm long, commencing at the pyloric sphincter and passing backwards, upwards, and rightwards to the neck of the gall-bladder, varying slightly in direction according to the degree of distension of the stomach. The proximal end of the super portion of the duodenum attaches to the distal end of the stomach through the pyloric sphincter, a sphincter muscle that closes tightly to prevent reflux from the duodenum to the stomach.

The descending portion of the duodenum is from about 7 to about 10 cm long, and extends from the neck of the gall-bladder on a level with the first lumbar vertebra downwards along the right side of the vertebral column as low as the upper border of the body of the fourth lumbar vertebra. The common bile duct and the pancreatic duct together perforate the medial side of descending portion of the duodenum obliquely 7 to 10 cm distal of the pyloric sphincter.

The horizontal portion of the duodenum is from about 5 to about 7.5 cm long beginning at the right side of the upper border of the fourth lumbar vertebra, passing from right to left, with a slight inclination upward.

The ascending portion of the duodenum is about 2.5 cm long, ascending along the left side of the aorta, as far as the level of the upper border of the second lumbar vertebra, abruptly turning forwards to become the jejunum.

The superior portion of the duodenum is somewhat movable, but the other portions are practically fixed and bound to neighboring viscera and the posterior abdominal wall by the peritoneum.

Embodiments of the present invention act, at least in part, by partially obstructing the lumen of the duodenum. In embodiments, such obstruction reduces the rate of passage of materials such as food through the duodenum. The reduced rate of passage of materials through the duodenum induces a faster onset and/or more intense and/or longer lasting perception of satiety in the subject induced by consumption of a given volume of food. The perception of satiety has a beneficial effect, for example a reduced perception of hunger that assists in maintaining a diet, reduced amount of food consumed and/or reduced amount of calories consumed.

As noted above, one of the reasons that a person overeats is a result of the delayed onset of satiety subsequent to food consumption. A person eats and fills the stomach until the stomach becomes distended to an extent that leads to the perception of satiety. There is, however, a time-lag between consumption of enough food to lead to a perception of satiety and the actual registration of the perception of satiety. Thus a person, especially a person who eats quickly, easily overeats consuming excess calories and consequently gaining weight. Apart from the direct weight gain, overeating and consequent stomach distension also causes the base volume of the stomach to increase and the gastric mechanoreceptors to become insensitive to small increases of stomach volume. A positive-feedback loop is generated where a person overeats because the indication of satiety from a distended stomach occurs only after satiety is reached, so that the person overeats, reducing the sensitivity of the satiety sensors and increasing the base stomach volume, so that the perception of satiety is delayed even further.

In embodiments of the present invention, the lumen of the duodenum is partially obstructed so that food enters the duodenum from the stomach through the pyloric sphincter in the usual way, but the rate of passage of the food past the obstruction is reduced, inducing a beneficial effect. Typical beneficial effects include a reduction of the amount of food consumed.

Although not wishing to be held to any one theory, it is believed that in embodiments food that is consumed and digested by the stomach passes the pyloric sphincter to enter the duodenum in the usual way. As the rate of food entering the duodenal volume upstream of the obstruction is substantially unchanged but the rate of food passing past the bottleneck in the duodenal lumen caused by the obstruction is reduced, a given volume of food fills the duodenum upstream of the obstruction, faster and for a longer time than otherwise, applying an outwards pressure on the duodenal walls. Further, in embodiments the partial obstruction effectively reduces the internal volume of the duodenum so that the duodenum fills up more quickly.

Presumably, the natural satiety inducing mechanisms of the duodenum related to duodenal mechanoreceptors respond as if a much greater volume of food has been consumed than otherwise. Apparently, embodiments of the present invention stimulate the natural mechanisms of the duodenum to induce desired beneficial effects.

Although not wishing to be held to any one theory, it is currently believed that in some embodiments of the invention, the feeling of satiety induced by the stimulation of duodenal chemoreceptors is increased due to the presence of a given volume of food for a longer time than otherwise. In some embodiments, the concurrent extra stimulation of the duodenal chemoreceptors and of the duodenal mechanoreceptors has a synergistic satiety-inducing effect.

According to the method of the present invention for the treatment of a condition related to an eating disorder, the lumen of a duodenum of a subject (whether a human or a non-human animal, but especially a human) suffering from the condition is partially obstructed, preferably so as to reduce the rate of passage of materials therethrough, leading to an effect beneficial for treating the condition. By treating the condition are included, but not limited to, curing the condition, treating the condition, preventing the condition, treating symptoms of the condition, curing symptoms of the condition, ameliorating symptoms of the condition, treating effects of the condition, ameliorating effects of the condition, and preventing results of the condition. Typical conditions from which a subject suffers include obesity, bulimia, eating disorders, overeating, diabetes-related obesity, BMI greater than 25 and metabolic syndrome. In embodiments, the partial obstructing comprises providing a device including a duodenum obstructing component and deploying the device so that the obstructing component is deployed in the lumen of the duodenum.

In embodiments, the deploying of the obstructing components is such that the partial obstruction commences in the superior portion of the duodenum. In embodiments, the deploying of the obstructing component is such that the partial obstruction substantially commences no more than about 5 cm from the pyloric sphincter.

In embodiments, the deployment of the obstructing component is substantially permanent, e.g., for the treatment of a chronic condition. In embodiments, the deployment of the obstructing component is temporary and the obstructing component is removed once a desired effect (e.g., sufficient weight loss) is achieved. The degree of partial obstruction and the location of deployment of the obstructing components depend on the severity and responsiveness of the condition to be treated. It will be appreciated that the location and degree of partial obstruction is preferably periodically adjusted based on, amongst other factors, the subject being treated, the severity of the condition and the judgment of the responsible health-care professional.

In embodiments, the obstructing component comprises at least one coiled section, in embodiments a coiled section comprising a conical coil shape. In embodiments, the coiled section comprises a coiled tube. In embodiments, the coiled section is configured to axially stretch. In embodiments, the coiled section is configured to axially bend.

In embodiments, the obstructing component is flexible or otherwise deformable. For example, in some embodiments where the obstructing component comprises a coiled section, the coiled section is a flexible coiled section, for example configured to axially stretch or axially bend. The coiled section effectively reduces the rate of passage of materials through the duodenum. Due to the flexibility of the coil, solid ingested materials are not permanently caught on the obstructing component but rather, by bending and/or stretching of the coil, are released before any clinically significant blockage of the duodenum occurs.

In embodiments, the obstructing component comprises an inflatable balloon including an internal volume.

In embodiments, the method further comprises anchoring the obstructing component in place in the duodenum.

In embodiments, the method further comprises holding the obstructing component in place at least in part by a component that passes through the pyloric sphincter into the duodenum from the stomach associated with the duodenum.

In embodiments, the method of the present invention further comprises varying (whether increasing or decreasing) the degree of the partial obstruction, and consequently the period of time and degree of stimulation of duodenal mechanoreceptors, when necessary. For example, in embodiments including an obstructing component comprising a coiled section such as discussed below, partial coiling or uncoiling of the coiled section or changing the length of the coil varies the degree of partial obstruction. For example, in embodiments including an obstructing component comprising an inflatable balloon including an internal volume, the introduction or removal of a fluid from the internal volume changes the conformation of the balloon and therefore varies the degree of partial obstruction.

In embodiments, the necessity of varying the degree of partial obstruction is determined manually, for example as a result of an evaluation of the degree of the beneficial effect. For example, periodically a health care professional such as the treating physician periodically (e.g., once a week, once a month, once in six months) examines the patient and evaluates the effect of the degree of partial obstruction of the duodenum. Generally, if a more intense or faster effect is desired, the degree of partial obstruction is increased. Generally, if a more moderate or slower effect is desired, the degree of partial obstruction is decreased

In embodiments, the necessity of varying the degree of the partial obstruction is determined by detection of an event of significance for varying the degree of the partial obstruction, for example in accordance with principles discussed in the PCT patent application of the Applicant published as WO 2006/035446.

For example, in embodiments, an event of significance for varying the degree of the partial obstruction is a physiological change indicative of, for example food ingestion or hunger, such as increased gastrointestinal tract activity, detection of muscle activity (e.g., of the stomach), pressure (e.g., caused by stomach contractions or resulting from food entering the stomach or esophagus), change of chemical composition such as pH (e.g., from the release of enzymes, acids or other gastric juices), body temperature and electrical currents in the vagus nerves or pancreas when a person is hungry or consuming food.

For example, in such embodiments when increased gastrointestinal activity indicative of food ingestion is detected, the degree of partial obstruction is increased so that the length of time and intensity of duodenal mechanoreceptor stimulation caused by food entering the duodenum from the stomach is increased, providing a longer and more intense perception of satiety. In embodiments, the varying of the degree of the partial obstruction is initiated a specified time subsequent to detection of the event, for example five minutes after the consumption of food is detected.

In embodiments, the detection of the event is automatically performed with the help of an event detector.

In embodiments, detection of an event is manual, that is to say, includes at least one step performed by a person, such as a caregiver, a health-care professional or the subject self. For example, the subject self anticipates a meal so increases the degree of partial obstruction.

In embodiments, the method of the present invention comprises a stimulation of the duodenum in addition to the stimulation caused by the partial obstruction. For example, in embodiments, the method of the present invention further comprises applying pressure to at least a portion of a luminal wall of the duodenum so as to directly stimulate duodenal mechanoreceptors, for example to induce a perception of satiety.

In embodiments, the method of the present invention further comprises, when necessary, applying at least one additional stimulus to the duodenum. In embodiments, the necessity of the application of the additional stimulus is periodic. For example, in embodiments, an additional stimulus is periodically applied with so that the person gets a continuous perception of satiety, e.g., once an hour.

In embodiments, the necessity of the application of the additional stimulus is determined by detection of an event of significance for application of the additional stimulus for example in accordance with principles discussed in the PCT patent application of the Applicant published as WO 2006/035446. For example, in embodiments, an event of significance for applying a stimulus is a physiological change indicative of, for example, food ingestion or hunger such as increased gastrointestinal tract activity. In embodiments, the application of the additional stimulus is initiated a specified time subsequent to detection of the event.

In embodiments, the detection of the event is automatically performed with the help of an event detector.

In embodiments, detection of an event is manual, that is to say, includes at least one step performed by a person, such as a caregiver, a health-care professional or the subject self. For example, the subject self anticipates a meal or feels hungry.

In embodiments, the additional stimulus comprises applying pressure for a period of time (generally no greater than about 60 minutes) to at least a portion of a luminal wall of the duodenum, preferably so that the applied pressure stimulates mechanoreceptors so as to induce a perception of satiety. As the pressure applied to the luminal walls in such an embodiment is applied only for a limited period of time and not continuously, the body does not get used to the applied pressure which would ultimately lead to desensitization to the stimulus. In embodiments, during the period of time the pressure is substantially constant. In embodiments, during the period of time the pressure is pulsatile.

In embodiments, the additional stimulus comprises electrically stimulating the duodenum for a period of time, generally for a period of time no greater than about 60 minutes preferably so that the electrical stimulation induces a perception of satiety. In embodiments the electrical stimulation is of nerves in the duodenum such as the vagus nerves.

In embodiments, the additional stimulus comprises administering a dose of a pharmaceutical composition comprising an active agent and a pharmaceutically acceptable carrier in the duodenum, preferably so that the administration of the pharmaceutical composition induces a perception of satiety. Administration is preferably spray administration of the active agent at the duodenal wall. As disclosed in U.S. patent application No. 60/903,289 of the Inventor, spray administration is an effective way of administering active agents, including peptide and peptide hormone active agents that function by interacting with chemoreceptors apparent on the luminal wall of the gastrointestinal tract.

In embodiments, the active agent is at least one agent selected from the group consisting of satiety agents and anti-food absorption drugs. Typical satiety agents useful in implementing the teachings of the present invention include peptide hormones, CCK, CCK receptor agonists, GLP-1, GLP-1 receptor agonists, PYY, PYY receptor agonists, oxyntomodulin, oxyntomodulin receptor agonists, analogs thereof and derivatives thereof (e.g. CCK-8) as taught in U.S. patent application No. 60/903,289 of the Inventor. A typical anti-food absorption drug is a lipase inhibitor.

An aspect of the present invention is also of a device useful for treatment of a condition related to an eating disorder and in embodiments useful in implementing at least some aspects of the method of the present invention, comprising: a) a duodenum obstructing component configured to partially obstruct the lumen of a duodenum in which deployed, preferably so as to reduce the rate of passage of materials through the duodenum; and b) an anchoring component configured to substantially maintain a position of the obstructing component inside a duodenum wherein deployed. It is important to note that in preferred embodiments of a device of the present invention, the obstructing component does not block entry of food into the duodenum but rather causes a given volume of food that enters the duodenum to induce a greater degree of satiety and/or to induce a perception of satiety for a longer period of time and/or to induce a perception of satiety faster than otherwise.

In embodiments, the device is provided with a marker (e.g., a feature or component) that is observable when deployed inside a body (for example by a medical imaging modality) and allows determination of the location of the obstructing component in the duodenum. In embodiments, a marker is radio-opaque and is observable using an X-ray emitting imaging modality. In embodiments, a marker is ultrasound opaque and is observable using an ultrasound imaging modality.

In embodiments, the anchoring component is substantially tensionless anchoring, that is does not apply a continuous pressure on bodily tissue, especially soft tissue, that may tear, distend or lead to other undesirable side-effects. In embodiments, the anchoring component is configured to substantially maintain the obstructing component no more than about 5 cm from a pyloric sphincter associated with the duodenum In embodiments of a device of the present invention, the anchoring component is configured to substantially maintain the obstructing component in the superior portion of a duodenum in which deployed.

In embodiments, the anchoring component is configured to apply outwards pressure to luminal walls of a gastrointestinal tract so as to maintain the obstructing component in the desired location in a duodenum, analogously to a stent.

In embodiments, at least part of the anchoring component is configured to pass through a pyloric sphincter from a stomach associated with the duodenum.

In embodiments, a device of the present invention comprises one or more anchoring components configured to penetrate into gastrointestinal tissue so as to maintain the obstructing component in the desired location in a duodenum. Anchors suitable for anchoring objects, in the gastrointestinal tract are well known in the art, see for example the PCT patent application published as WO2006/111961. As is known to one skilled in the art, implanted devices such as anchors are preferably implanted tension-free (that is with no substantially continuous pressure) to prevent pain, tearing of surrounding tissue, migration and/or release of the anchor. Thus, it is preferred that an anchor anchoring an obstructing component of the present invention be tension free, for example, comprises a loose suture or loose staple.

In embodiments, the obstructing component is configured to have at least two conformations, a first conformation and a second conformation providing different degrees of partial duodenal obstruction.

In embodiments, a device of the present invention further comprises a conformation-determining component configured to transform the obstructing component from the first conformation to the second conformation while the obstructing component is deployed within a duodenum. In embodiments, the conformation determining component is configured to reversibly transform the obstructing component from the first conformation to the second conformation while the obstructing component is deployed within a duodenum. For example, in embodiments, the conformation-determining component includes an internal volume (e.g., comprises a balloon) and the transformation is effected by a process including transport of fluid into and/or out of the internal volume. For example, in embodiments, the conformation-determining component comprises a heat-sensitive element and the transformation is effected by a process including changing the temperature of the heat-sensitive element.

In embodiments, the conformation-determining component is configured for manual manipulation, that is to say, the conformation of the obstructing component is adjustable by an intervention that includes at least one step performed by a person, such as a caregiver, a health-care professional or the subject in which the device is deployed self.

In embodiments, a device of the present invention further comprises an actuator configured to trigger the conformation-determining component to transform the obstructing component from the first conformation to the second conformation. In embodiments, a device of the present invention further comprises a timer functionally associated with the actuator. In embodiments, the timer is configured to activate the actuator to trigger the conformation-determining component for a specified period of time. In embodiments, the timer is configured to periodically activate the actuator to trigger the conformation-determining component.

In embodiments, a device of the present invention further comprises an event detector functionally associated with the actuator, so that the actuator triggers the conformation-determining component as a consequence of detection of an event of significance for changing the conformation. In embodiments, the event detector comprises an electrode configured for deployment in a body. In embodiments, the event detector is configured to detect a physiological change (e.g., increased gastrointestinal tract activity) indicative of a member of the group consisting of food ingestion and hunger. In embodiments, a device of the present invention further comprises a timer functionally associated with the actuator and with the event detector. In embodiments, the timer is configured to activate the actuator to trigger the conformation-determining component a specified period of time subsequent to detection of an event by the event detector.

In embodiments, a device of the present invention further comprises a component configured to apply a stimulus to a duodenum in which deployed in addition to the stimulus caused by the partial obstruction of a duodenum by the obstructing component. In embodiments, the component configured to apply an additional stimulus is substantially the obstructing component. In embodiments, the component configured to apply an additional stimulus is distinct from the obstructing component.

In embodiments, the component is configured to apply the additional stimulus substantially continuously.

In embodiments, a device of the present invention further comprises an actuator configured to trigger the additional stimulus applying component to apply an additional stimulus to the duodenum. In embodiments, a device of the present invention further comprises a timer functionally associated with the actuator. In embodiments, the timer is configured to activate the actuator to trigger the additional stimulus applying component for a specified period of time. In embodiments, the timer is configured to periodically activate the actuator to trigger the additional stimulus applying component. In embodiments, a device of the present invention further comprises an event detector functionally associated with the actuator, so that the actuator triggers the additional stimulus applying component as a consequence of detection of an event of significance for applying the additional stimulus. In embodiments, the event detector comprises an electrode configured for deployment in a body. In embodiments, the event detector is configured to detect a physiological change (e.g., increased gastrointestinal tract activity) indicative of a member of the group consisting of food ingestion and hunger. In embodiments, a device of the present invention further comprises a timer functionally associated with the actuator and with the event detector. In embodiments, the timer is configured to activate the actuator to trigger the additional stimulus applying component a specified period of time subsequent to detection of an event by the event detector.

In embodiments, a device of the present invention further comprises a mechanoreceptor-stimulating component as an additional duodenum stimulating component, the mechanoreceptor-stimulating component configured to apply an outwards pressure to at least a portion of a luminal wall of a duodenum in which deployed. In embodiments, the mechanoreceptor-stimulating component is configured to have at least two conformations, a first conformation and a second conformation providing different degrees of the outwards pressure. In embodiments, a surface of the mechanoreceptor-stimulating component configured to contact the portion of the luminal wall includes duodenal wall stimulating protuberances such as grooves, strips, studs or spikes. Typically, such protuberances are between about 50 micrometers and 3 millimeters in height.

In embodiments, a device of the present invention further comprises a mechanoreceptor-stimulating component conformation determiner configured to transform the mechanoreceptor-stimulating component from the first conformation to the second conformation while the mechanoreceptor-stimulating component is deployed within a duodenum. In embodiments, the mechanoreceptor-stimulating component conformation determiner is configured to reversibly transform the mechanoreceptor-stimulating component from the first conformation to the second conformation while the mechanoreceptor-stimulating component is deployed within a duodenum. For example, in embodiments, the mechanoreceptor-stimulating component conformation determiner includes an internal volume and the transformation is effected by a process including transport of fluid into and/or out of the internal volume. For example, in embodiments, the mechanoreceptor-stimulating component conformation determiner comprises a heat-sensitive element and the transformation is effected by a process including changing the temperature of the heat-sensitive element.

In embodiments, a device of the present invention further comprises an electrical stimulator component as an additional duodenum stimulating component configured to electrically stimulate at least a portion of a luminal wall of a duodenum in which deployed. In embodiments, the electrical stimulator is configured to stimulate nerves in a duodenum in which the device is deployed, such as the vagus nerves, in order to induce a perception of satiety.

In embodiments, a device of the present invention further comprises an active agent dispensing component configured to administer a dose of a pharmaceutical composition comprising an active agent and a pharmaceutically acceptable carrier. In embodiments, the active agent dispensing component comprises a sprayer configured to direct a spray towards a luminal wall of a duodenum in which deployed. In embodiments, a device of the present invention further comprises an active agent reservoir functionally associated with the active agent dispensing component and also a pressure generator configured to transport a composition held in the active agent reservoir to be dispensed through the active agent dispensing component.

In embodiments, a device of the present invention further comprises a power supply unit for providing power for operation of other components of the device. In embodiments, the power supply unit is configured for implantation in a body, e.g., in the gastrointestinal tract, the stomach or subcutaneous implantation. In embodiments, the power supply unit comprises a power storage unit. In embodiments, a power storage unit is rechargeable. In embodiments, a power supply unit comprises a power generation unit.

In embodiments of a device of the present invention, an obstructing component comprises at least one coiled section. In embodiments, one such coiled section is a coiled elongated element having a cross sectional area of no more than about 0.25 cm², no more than about 0.1 cm² and even no more than about 0.04 cm², e.g., in embodiments having a solid cross section such as a wire or ribbon. In embodiments, the cross section of the elongated element is, for example, round or square. In embodiments, for example, the cross section of the elongated element has a greater and a lesser dimension, e.g. is oval or rectangular.

In embodiments, the axial length of at least one such coiled section is at least about 1 cm. In embodiments, the axial length of at least one such coiled section is no more than about 5 cm. In embodiments, the distance between any two loops of such a coiled section is at least about 0.5 cm. In embodiments, at least one such coiled section comprises a conical coil shape.

In embodiments, at least one such coiled section comprises a coiled tube including an axial channel passing therethrough.

In embodiments, the coiled section comprises an open tube with an open-ended axial channel running therethrough, for example to allow deployment of the coiled section in a duodenum with the help of a delivery guide wire that straightens the coiled section as discussed below.

In embodiments, at least one such coiled section comprises a close-ended tube with an elongated axial internal volume inside the coiled section. In embodiments, a device of the present invention further comprises a pressure generator configured to force fluid into and/or out of the internal volume, so as to change a conformation of the coiled section as discussed below. In embodiments, a device of the present invention further comprises a fluid reservoir functionally associated with the pressure generator. In embodiments, such a fluid reservoir is configured for implantation in a body, e.g., in the gastrointestinal tract, the stomach or subcutaneous implantation.

In embodiments, such a coiled section comprises a heat-sensitive element configured to change conformation upon changing of temperature, e.g., to a larger or smaller radius coil or to a shorter or longer coil. In embodiments, the heat sensitive element comprises a heat sensitive shape memory alloy. In embodiments, a device of the present invention further comprises a heating element functionally associated with the heat-sensitive element so as to function as a conformation determining component by heating the heat sensitive element. In embodiments, a device of the present invention further comprises a power supply configured to supply power to the heating element

In embodiments of a device of the present invention, an obstructing component comprises an inflatable balloon including an internal volume. In embodiments, such an inflatable balloon is provided with at least a portion of a wall that is elastic so that a fluid introduced into the internal volume causes the wall to expand outwards changing the conformation of the balloon. In embodiments, a device of the present invention further comprises a pressure generator configured to force fluid into and/or out of the internal volume of the balloon, so as to change a conformation of the balloon. In embodiments, a device of the present invention further comprises a fluid reservoir functionally associated with the pressure generator, so that the pressure generator is configured to transport fluid from the fluid reservoir into the internal volume of the balloon and/or from the internal volume of the balloon into the fluid reservoir. In embodiments, such a fluid reservoir is configured for implantation in a body, e.g., in the gastrointestinal tract, the stomach or subcutaneous implantation.

In embodiments, an inflatable balloon is elongated and substantially straight (sausage-shaped). In embodiments, the inflatable balloon is elongated and configured to have a coiled conformation when partially inflated. In such embodiments, the diameter of the balloon in an inflated conformation typically has a cross section of less than about 1 cm and even less than about 0.5 cm.

In embodiments, an inflatable balloon is annular with a balloon lumen and an external diameter. In embodiments, such an inflatable balloon is configured to have a smaller diameter balloon lumen with increased inflation. In embodiments, such an inflatable balloon is configured to have a greater external diameter with increased inflation.

An aspect of the present invention is a method of treatment of a condition related to an eating disorder, comprising: a) providing a device of the present substantially as described above; b) deploying an obstructing component of the device in the lumen of a duodenum of a subject suffering from the condition so as to partially obstruct the lumen of the duodenum; and c) using an anchoring component of the device to substantially maintain a position of the obstructing component inside the duodenum; thereby reducing the rate of passage of materials through the duodenum, leading to an effect beneficial for treating the condition.

The principles of the method and the device of the present may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

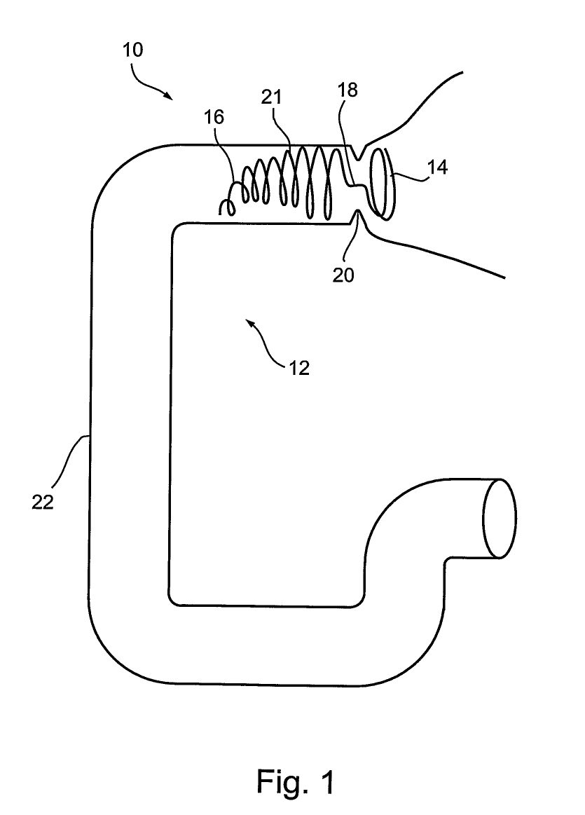

An embodiment of a device of the present invention, device 10 is depicted in FIG. 1 deployed in a duodenum 12. Device 10 is substantially a single body-temperature shape-memory Nickel-Titanium alloy (Nitinol) wire with a 2 mm diameter (available from, for example, Endosmart Gesellschaft für innovative Medizintechnik mbH, Stutensee, Germany) covered with an inert polymer coating of polyfluorohydrocarbon (e.g., polytetrafluorethylene available from E. I. du Pont de Nemours and Company Wilmington, Del., USA).

At low temperatures (e.g., less than about 15° C.) device 10 is in a first conformation, that of a substantially straight wire.

At body temperatures (e.g., greater than about 35° C.) device 10 is in a second conformation depicted in FIG. 1. In the second conformation, device 10 is coiled so as to have three distinct sections: an anchoring section 14, a duodenum obstructing section 16 constituting a duodenum obstructing component of device 10, and a connecting section 18 connecting between anchoring section 14 and obstruction section 16 and constituting, together with anchoring section 14, a portion of an anchoring component of device 10.

Anchoring section 14 is substantially a cylindrical coil consisting of 1.5 loops around the longitudinal axis of device 10 at the proximal end of device 10. Anchoring section 14 is greater than about 3 cm in diameter, sufficiently wide so as not to easily pass through pyloric sphincter 20.

Duodenum obstructing section 16 is substantially a conical coil around the longitudinal axis of device 10 tapering towards the distal end of device 10. The proximal end of obstruction section 16 is greater than about 3 cm in diameter, sufficiently wide so as not to easily pass through pyloric sphincter 20. Although, in embodiments, the diameter of obstruction section 16 is significantly greater than 3 cm in diameter, in device 10 depicted in FIG. 1 the diameter of obstruction section 16 is generally less than 4 cm so as not to apply significant outwards pressure on the luminal walls of duodenum 12. The distance between any two loops of obstruction section 16 is relatively large, generally greater than about 0.5 cm. The length of obstruction section 16 of device 10 is approximately 3 cm. Duodenum obstructing section 16 is flexible, both by axial bending and axial stretching, in order to allow solid materials such as incompletely chewed food from causing blockage of the duodenum.

Connecting section 18 is a straight section of wire substantially coaxial with the longitudinal axis of device 10 that connects between anchoring section 14 and duodenum obstructing section 16. The length of connecting section 18 determines, in a large part, the location where obstruction section 16 is deployed in duodenum 12. In device 10, the length of connecting section 18 is about 1 cm so that obstruction section 16 is deployed commencing about 1 cm from pyloric sphincter 20.

For deployment in duodenum 12, device 10 is placed inside a deployment sleeve (e.g., a flexible tube such as known in the art of endoscopes) having a bore with a diameter greater than that of the diameter of the wire making up device 10 so that device 10 is forced into and remains in a substantially straight conformation. The deployment sleeve enclosing device 10 is maneuvered, for example with the help of a gastroscope, past pyloric sphincter 20 and into duodenum 12. With the help of a plunger, device 10 is pushed out of the deployment sleeve, distal end first. As device 10 emerges from the deployment sleeve, device 10 is no longer constrained and is heated by the body of the subject so that device 10 transforms to the second conformation. Specifically, device 10 coils in duodenum 12 to form the conical coiled shape of obstruction section 16. When the section of device 10 that corresponds to connecting section 18 emerges from the deployment sleeve, the deployment sleeve is positioned in the stomach proximal to pyloric sphincter 20 so that device 10 coils to form anchoring section 14 proximal to pyloric sphincter 20. It is important to note that although deployment of device 10 with the help of a gastroscope is described above, in embodiments a device of the present invention is deployed, substantially analogously, transcutaneously or through another mode.

Subsequent to deployment, device 10 straddles pyloric sphincter 20, so that pyloric sphincter 20 is flanked by anchoring section 14 in the stomach and obstruction section 16 in superior portion 21 of duodenum 12 while connecting section 18 passes through pyloric sphincter 20. Anchoring section 14 maintains obstruction section 16 in place in a substantially tensionless fashion, resting against parts of the gastrointestinal tract lumen, but with little, if any, pressure or strain.

When a subject consumes food, the food enters the stomach and is partially digested in the usual way. The partially digested food passes through pyloric sphincter 20 into superior portion 21 of duodenum 12. In superior portion 21 of duodenum 12 the partial obstruction of the lumen of duodenum 12 caused by obstruction section 16 of device 10 reduces the rate of passage of the food through duodenum 12 by a number of mechanisms including by reducing the effective cross section of the lumen of duodenum 12 so as to produce a bottle neck and also by trapping some of the food.

Since the rate of passage of the food past obstruction section 16 and through duodenum 12 is reduced, a given volume of food consumed passes more slowly through duodenum 12. The same volume of food fills superior portion 21 of duodenum 12 more and thus applies a greater outwards pressure to the duodenum walls. Thus, a given volume of food more quickly applies a greater outwards pressure to the duodenum walls, more quickly activating mechanoreceptors in superior portion 21 of duodenum 12 so that a perception of satiety is induced more quickly. Further, the time for the food to pass through duodenum 12 is longer so that the perception of satiety lasts longer. Since the perception of satiety is induced more quickly, to a greater extent and for longer with a lesser volume of consumed food, a subject in whose duodenum 12 device 10 is deployed consumes less food and/or fewer calories.

Occasionally, the flow of food through duodenum 12 or peristaltic motion of the gastrointestinal tract pushes device 10 downstream. Anchoring section 14 is pulled against pyloric sphincter 20 preventing the passage of device 10 entirely into duodenum 12 and thus substantially maintaining the position of obstruction section 16 in duodenum 12. Under certain conditions the intermittent pressure of anchoring section 14 against pyloric sphincter 20 stimulates pyloric mechanoreceptors to induce a perception of satiety.

Device 10 as described above leads to stimulation of duodenum 12 primarily by partially obstructing the lumen of duodenum 12 so as to reduce the rate of passage of material such as food through duodenum 12. In embodiments, a device of the present invention is configured to apply a stimulus to a duodenum in which deployed in addition to the stimulus caused by partial obstruction of the duodenal lumen.

In a non-depicted embodiment substantially similar to device 10 in FIG. 1, an obstruction section 16 is configured to press against the duodenal walls to act as a mechanoreceptor-stimulating component thus applying an additional stimulus to a duodenum in which deployed. In such an embodiment, the diameter of the widest portion of an obstruction section 16 is, in an unconstrained state, greater than the luminal diameter of a duodenum 12 in which deployed, e.g., greater than about 4 cm in diameter. Preferably, the wire from which such a device is fashioned is not round as in device 10, but rather has a greater and a lesser dimension (e.g., 5 mm wide and 1 mm thick), like a ribbon or band. When deployed in a duodenum 12, the diameter of the widest portion of obstruction section 16 is constrained by the luminal duodenal walls and therefore applies a substantially continuous outwards pressure on the luminal walls that varies in intensity as a result of peristaltic contractions of the duodenum, stimulating duodenal mechanoreceptors to induce a perception of satiety. The device is configured so that the surface contacting the duodenal wall is the greater dimension so that the pressure is distributed over a large area of and thus avoids penetration of the duodenal wall. In such embodiments where an obstruction section 16 is configured to act directly as a mechanoreceptor-stimulating component, the outer surface of obstruction section 16 which contacts the luminal wall of the duodenum is optionally provided with wall-stimulating features, for example by roughening or by including features such as grooves, strips, studs or spikes. Typically, such features are between about 50 micrometers and 3 millimeters in height. It is important to note that in such embodiments, obstruction section 16 also acts as an anchoring component by applying outwards pressure to the luminal walls of duodenum 12 which helps to maintain the obstruction section 16 in the desired location in a duodenum 12.

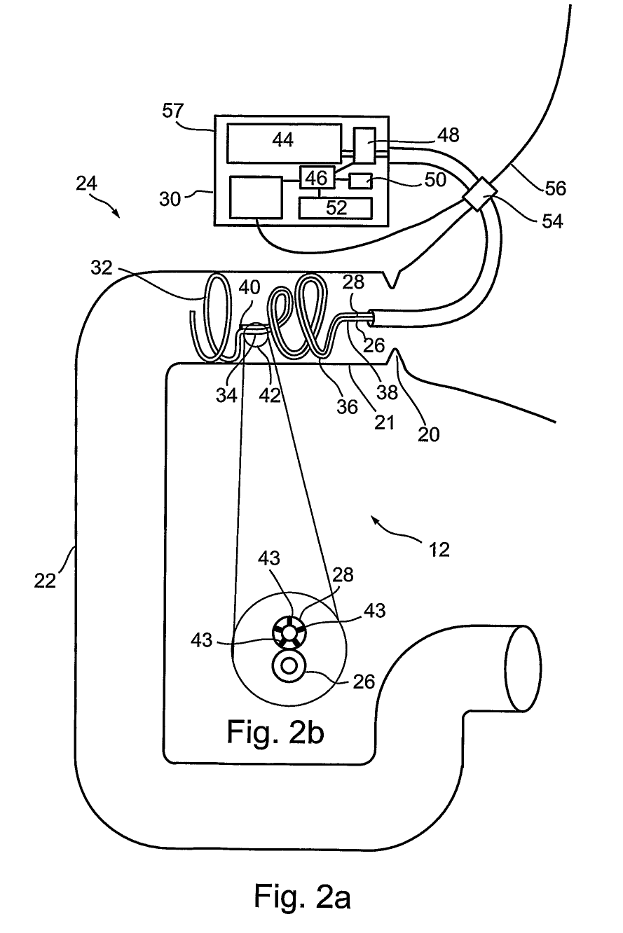

An additional embodiment of a device of the present invention, device 24 is depicted in FIGS. 2A and 2B with a duodenum obstructing component 36 deployed inside a duodenum 12. Device 24 is configured for treatment of a condition relating to an eating disorder by providing three different satiety-inducing stimuli to duodenum 12. Device 24 is configured to partially obstruct the lumen of a duodenum in which deployed and is configured to directly stimulate duodenal mechanoreceptors by applying an outwards pressure to portions of the luminal wall of the duodenum. Device 24 further comprises an active agent dispensing component configured to administer a dose of a pharmaceutical composition comprising an active agent and a pharmaceutically acceptable carrier, the active agent dispensing component comprising a sprayer 42 configured to direct a spray towards a luminal wall of a duodenum in which deployed, an active agent reservoir 44 functionally associated with the active agent dispensing component and also a pressure generator 48 configured to transport a composition held in active agent reservoir 44 to be dispensed through the active agent dispensing component.

Device 24 comprises two parallel coiled tubes: an obstruction tube 26 and an active agent feeder tube 28, both functionally associated with control unit 30.

Obstruction tube 26 is a flexible polymer tube (such as a Cook Nasal Biliary Drainage Sets, ENBD-6-Liguory, GPN G21725 with a 2 mm outer diameter and 1 mm diameter open axial channel running through the entire length thereof) fashioned to have four distinct section: distal anchoring section 32, active agent feeder support section 34, duodenum obstructing section 36 and proximal anchoring section 38.

Distal anchoring section 32 is fashioned so as to constitute a parallel walled coil of 1.5 turns having an axial length of about 1 cm and has an unconstrained diameter greater than that of duodenum 12. When deployed in duodenum 12, the diameter of distal anchoring section 32 is constrained by the luminal duodenal walls and therefore applies a substantially continuous outwards pressure on the luminal walls that varies in intensity as a result of peristaltic contractions of duodenum 12, thus stimulating duodenal mechanoreceptors to induce a perception of satiety. Thus, in device 24, a component distinct from the obstructing component applies a stimulus to duodenum 12. Further, distal anchoring section 32 constitutes a component of an anchoring component of device 24 by applying outwards pressure to the luminal walls of duodenum 12, which helps to maintain duodenum obstructing section 36 in the desired location in a duodenum 12. Further, distal anchoring section 32 helps maintain active agent sprayer 42 (at the distal end of tube 28) substantially coaxially in the lumen of duodenum 12, as discussed below.

Active agent feeder support section 34 is substantially a non-coiled section of polymer tube 26 connecting between distal anchoring section 32 and duodenum obstructing section 36. Active agent feeder support section 34 helps maintain active agent sprayer 42 substantially coaxially with the lumen of duodenum 12.

Duodenum obstructing section 36 is substantially a conical coil around the longitudinal axis of obstruction tube 36 tapering towards the distal end of obstruction tube 26 having about 2 turns, a length of about 2 cm and a greatest unconstrained diameter at a proximal end that is greater than that of duodenum 12. When deployed in duodenum 12, the diameter of the proximal end of duodenum obstructing section 36 is constrained by the luminal duodenal walls and therefore applies a substantially continuous outwards pressure on the luminal walls that varies in intensity as a result of peristaltic contractions of duodenum 12, thus stimulating duodenal mechanoreceptors to induce a perception of satiety. Further, duodenum obstructing section 36 constitutes a component of the anchoring component of device 24 by applying outwards pressure to the luminal walls of duodenum 12, which helps to maintain duodenum obstructing section 36 in the desired location in a duodenum 12. Further, duodenum obstructing section 36 helps maintain active agent sprayer 40 substantially coaxially in the lumen of duodenum 12.

Proximal anchoring section 38 is the non-coiled flexible proximal end of obstruction tube 26 that is fixed to control unit 30 so as to help maintain duodenum obstructing section 36 in the desired location in duodenum 12 and thus constitutes a component of anchoring component of device 24.

Active agent feeder tube 28 is a hollow tube (such as a Cook Nasal Biliary Drainage Sets, ENBD-6-Liguory, GPN G21725 with a 2 mm outer diameter and 1 mm channel therethrough) parallel with obstruction tube 26 passing from control unit 30 to the distal end of active agent feeder support section 34 of obstruction tube 26. At the distal tip of feeder tube 28 and blocking the channel of feeder tube 28 is a stainless steel plug 40 while the distal 1 cm of feeder tube 28 is perforated with a plurality of perforations 43 arrayed about the axis of feeder tube 28 constituting nozzles (see FIG. 2B, a cross section of feeder tube 28 and obstruction tube 26) so that the distal end of feeder tube 28 is a sprayer for spray delivery of active agents as taught U.S. patent application No. 60/903,289 of the Inventor. Plug 40 is detectable by various medical imaging modes such as ultrasound and X-rays and therefore constitutes a marker for determining the position of the various parts of device 24 and especially nozzle 43 when deployed in the body of a subject.

Control unit 30 is analogous to a control unit of a composition dispensing device as described in U.S. patent application No. 60/903,289 of the Inventor and includes composition reservoir 44, a controller 46, pressure generator 48, a timer 50, power storage unit 52 all held inside a casing 57, and an event detector 54.

Reservoir 44 comprises a chamber for holding a pharmaceutical composition and is in fluid communication with feeder tube 28 through pressure generator 48. In embodiments, reservoir 44 is provided with a charging port allowing charging of the chamber when control unit 30 is implanted inside a body.

Pressure generator 48 is a pump that, when receiving power, forces a composition held in reservoir 44 into feeder tube 28 and out through nozzles 43 as a spray.

Event detector 54 is a gastric activity sensor (such implemented in the Tantalus™ System (Metacure NV, MetaCure N.V., Curacao, Netherlands Antilles) is implanted in gastric wall 56 and is configured to detect an electrical activity event in the gastric wall 54 to monitor gastric neural and muscle activity indicative of hunger or food ingestion. When such an event is detected, event detector 54 transmits the fact of detection to controller 46. Event detector 54 serves an additional function, defining a passage through which obstruction tube 26 and feeder tube 28 pass through the wall of stomach 56, but preventing the passage of gastric fluids from the stomach into the abdomen.

Device 24 is also provided with power storage unit 52 as a power supply unit for providing power for operation of other components of device 24. In device 24, power storage unit 52 is a rechargeable battery functionally associated with components of device 24 that require power.

Controller 46 comprises a populated circuit board with appropriate electronic components and is functionally associated with timer 50 to constitute an actuator and connects between power storage unit 52 and pressure generator 48. Amongst other functions, controller 46 is configured that upon receipt of a signal that an event is detected from event detector 54, controller 46 allows power to pass from power storage unit 52 to pressure generator 48 for a specified time with reference to timer 50.

Casing 57 is configured for subcutaneous implantation in the body. In addition to the function of encasing and protecting the other components of control unit 30 from the conditions in the body, casing 57 also constitutes a component of an anchoring component of device 24, helping maintain obstruction section 36 of device 24 in place in duodenum 12.

For deployment in duodenum 12, a flexible but straight guide wire (as is known in the art of endoscopy and catheters) is placed through the bore of obstruction tube 26 so that obstruction tube 26 and feeder tube 28 are forced into and remain in a substantially straight conformation. Event detector 54 is implanted in stomach wall 56. Obstruction tube 26 and feeder tube 28 are percutaneously introduced into the body and guided through a hole in event detector 54 to enter the stomach, for example with the help of a gastroscope. Obstruction tube 26 and feeder tube 28 are then guided past pyloric sphincter 20 into duodenum 12. The guide wire is carefully withdrawn outwards from the bore of obstruction tube 26 so that the distal end of obstruction tube 26 coils to form distal anchoring section 32 and duodenum blocking section 36 inside duodenum 12. The withdrawal of the guide wire is performed so that blocking section 36 commences about 2 cm distal to pyloric sphincter 20 and so that sprayer 42 is positioned so as to direct a spray at the luminal wall of duodenum 12 including parts of superior section 21 of duodenum 12. Distal anchoring section 32 is positioned so that active agent feeder support section 34 maintains sprayer in location near the longitudinal axis of duodenum 12. The positioning of obstruction tube 26 and feeder tube 28 so that the withdrawal of the guide wire will lead to correct deployment of distal anchoring section 32 is easily performed with reference to the location of plug 40 which is detectable with the help of medical imaging modalities such as ultrasound and X-rays as described above. Once deployed, feeder tube 28 is functionally associated with an outlet of pressure generator 48 and the proximal end of obstruction tube 26 is secured to casing 57 of control unit 30. Control unit 30 is implanted subcutaneously and composition reservoir 44 is charged with a pharmaceutical composition including a satiety agent, e.g., CCK-8, as described U.S. patent application No. 60/903,289 of the Inventor.

Once deployed, the proximal end of obstruction section 36 and distal anchoring section 32 apply a substantially continuous outwards pressure on the luminal walls that varies in intensity as a result of peristaltic contractions of duodenum 12, continuously inducing a perception of satiety by pressing outwards on the luminal walls of duodenum 12, thus constituting mechanoreceptor-stimulating components. Analogously to the discussed above, obstruction section 36 and distal anchoring section 32 are optionally provided with wall-stimulating features, such as roughening, grooves, strips, studs or spikes.

In addition, device 24 is configured to function in accordance with the teachings of U.S. patent application No. 60/903,289 of the Inventor by administering the pharmaceutical composition held in reservoir 44 by spraying the composition at the luminal walls of duodenum 12. Specifically, device 24 is configured to provide an additional stimulus to duodenum 12 by automatically administering a dose of an active agent when necessary, by spraying the pharmaceutical composition held in reservoir 44 through sprayer 40 at the luminal wall of duodenum 12.

The subject in which device 20 is deployed goes about life in the usual way. When the subject becomes hungry or begins to ingest food, various physiological changes (e.g., increased gastrointestinal tract activity) occur that are automatically detected by event detector 54. The fact of detection of the event is transmitted by event detector 54 to controller 46. Controller 46 triggers pressure generator 48 by allowing power from power storage unit 44 to pass to pressure generator 48 for a specified period of time as determined by timer 50. The size of the dose of active agent administered is determined, in part, by the specified period of time. Pressure generator 48 pumps the pharmaceutical composition from reservoir 44 through active agent feeder tube 28 to sprayer 42. The pressure at which pressure generator 48 pumps the composition into sprayer 42 forces the composition out through nozzles 43 at the luminal wall of duodenum 12. The active agent in the composition interacts with satiety chemoreceptors found on the luminal wall of duodenum 12, leading to a perception of satiety in the subject. The perception of satiety causes the person to consume a reduced amount of food.

When the reduced amount of food consumed enters duodenum 12 from the stomach, the fact that obstruction section 36 of device 24 partially obstructs the lumen of duodenum 12 induces a relatively quick onset, relatively intense and relatively long-duration perception of satiety as described above for device 10.

In embodiments, controller 46 is configured to trigger pressure generator 48 immediately upon or after a predetermined time upon detection of an event of significance to the administration of the composition. In a preferred embodiment, pressure generator 48 is triggered immediately and for a short time (e.g., a few second) so as to “blunt” the perception of hunger as soon as detected to assist the subject in reducing the amount of food consumed as demonstrated in U.S. patent application No. 60/903,289 of the Inventor.

Periodically, the correct location of sprayer 42 and obstruction section 36 is confirmed non-invasively by observing the location of stainless steel plug 40. If necessary, a guide wire is placed through the channel in obstruction tube 26 so as to straighten distal anchoring section 32 and obstruction section 36, allowing obstruction section 36 and sprayer 42 to be moved.

As described above, controller 46 constitutes an actuator that is configured to trigger administration of a pharmaceutical composition when needed, where the need is detection of event of significance for the administration of the composition. In embodiments, controller 46 is configured (additionally or alternately) to trigger administration of the pharmaceutical composition when needed, where the need is periodic for example to maintain a perception of satiety over a period of time. For example, in such an embodiment controller 46 periodically administers a dose of pharmaceutical composition with reference to timer 50, for example once every three hours.

As noted above, device 24 depicted in FIG. 2 is provided with three different modes of duodenal stimulation: the substantially continuous stimulation of mechanoreceptors by distal anchoring section 32 and the proximal portion of obstruction section 36, by the administration of a pharmaceutical composition through sprayer 42 and by partially obstructing the lumen of duodenum 12 with obstructing component 36.

A non-depicted embodiment of a device of the present invention similar to device 24 further comprises an electrical stimulator component as an additional duodenum stimulating component configured to electrically stimulate at least a portion of a luminal wall of a duodenum in which deployed. In embodiments, the electrical stimulator is configured to stimulate nerves in a duodenum in which the device is deployed, such as the vagus nerves in order to induce a perception of satiety. For example, in some such embodiments, protruding from the outwardly facing walls of distal anchoring section 32 and/or of the proximal portion of obstruction section 36 are gold electrodes that are in electrical communication with controller 46 which is configured to provide a fourth mode of duodenal stimulation. In such embodiments, the surfaces of the coiled sections including the electrodes, press against and even penetrate somewhat into the duodenal wall. Electrical current passed through electrodes, or a potential difference between at least two such electrodes is then used, for example, for vagal nerve stimulation to provide a perception of satiety. The use of such electrodes to electrically stimulate the duodenum is discussed in U.S. patent application No. 60/903,289 of the Inventor, for example to electrically stimulate nerves in the duodenum. Preferably the electrodes are also configured to function as physical wall-stimulating features as discussed above.

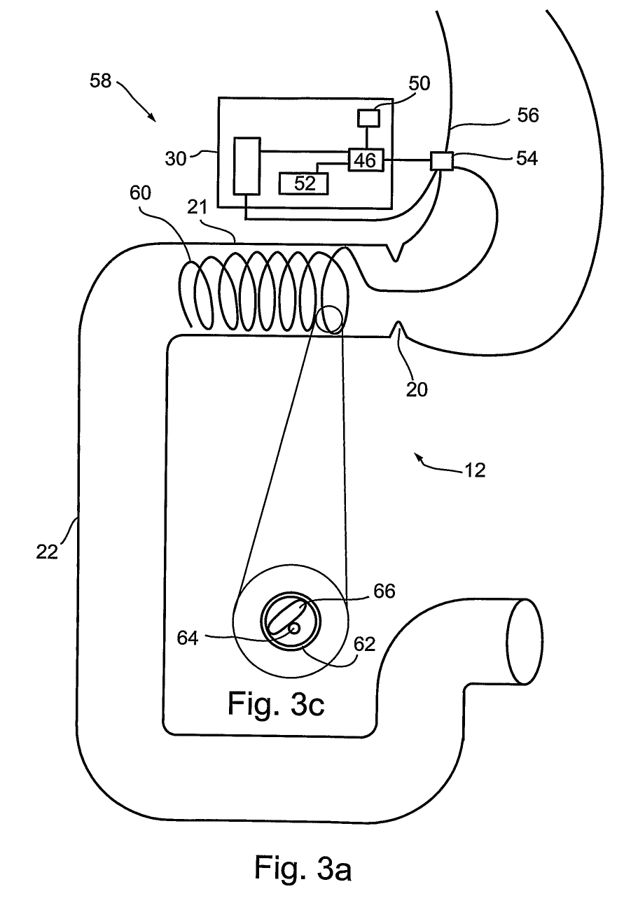

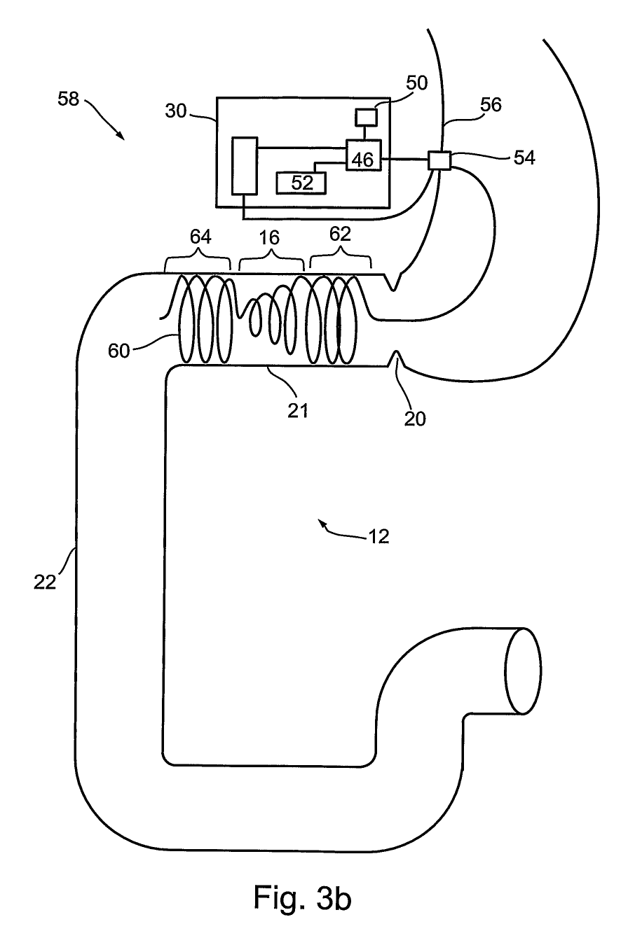

An additional embodiment of a device of the present invention, device 58, is depicted in FIGS. 3A, 3B and 3C. Device 58 comprises a control unit 30, an event detector 54 and a duodenum stimulating coil 60 having a first rest conformation depicted in FIG. 3A and a second obstructing conformation depicted in FIG. 3B.

Control unit 30 of device 58 is a subcutaneously implantable component substantially similar to control unit 30 of device 24 depicted in FIG. 2A and includes a power supply unit 52, a timer 50 and a controller 46. Controller 46 is functionally associated with event detector 54 which is substantially similar to event detector 54 of device 24 depicted in FIG. 2A.

Stimulating coil 60 comprises a flexible polymer tube 62 of a material resistant to the conditions of the gastrointestinal tract (e.g., a fluorocarbon polymer such as polytetrafluoroethylene available from E. I. du Pont de Nemours and Company Wilmington, Del., USA) which encases a heating element 64 and a heat sensitive element 66. Heating element 64 comprises, for example, a high resistance wire (e.g., Nichrome) that heats up when current passes therethrough. Heat sensitive element 66 is substantially a single, continuous high-temperature shape-memory Nickel-Titanium alloy (Nitinol) ribbon 3 mm broad and 1 mm thick (available from, for example, Endosmart Gesellschaft für innovative Medizintechnik mbH, Stutensee, Germany). Heat sensitive element 66 is configured to have two conformations. At body temperature heat-sensitive element 66 is in a first rest conformation comprising a 4 cm long/2 cm diameter coil 60, the loops of coil 60 spaced approximately 0.5 cm apart (FIG. 3A). At a higher temperature, heat-sensitive element 66 is in a second obstruction conformation where coil 60 adopts three distinct sections, two 2 cm long cylindrical coiled sections at either end, a proximal mechanoreceptor stimulating section 62, distal mechanoreceptor stimulating section 64 and a 2 cm long distally-tapering conical coiled obstruction section that constitutes a duodenum obstructing component of device 58 (FIG. 3B). Heat-sensitive element 66 and heating element 64 together comprise components of a mechanoreceptor-stimulating component conformation determiner and of an obstructing component conformation determiner and function together to reversibly transform coil 60 from the first conformation to the second conformation while deployed within a duodenum.

Control unit 30 together with the non-coiled proximal section of coil 60 together constitute an anchoring component to substantially maintain the position of the obstruction section of coil 60 in the proximity of pyloric sphincter 20 in duodenum 12.

Tube 62, heating element 64, heat sensitive element 66 and controller 30 together comprise a conformation-determining component of device 58 and are configured to transform the obstructing component of device 58, coil 60, from the first conformation to the second conformation while coil 60 is deployed within a duodenum. Controller 46 constitutes an actuator that triggers the conformation determining component to initiate the transformation of coil 60 from the first conformation to the second conformation.

Deployment of device 58 is analogous to deployment device 24 depicted in FIG. 2 and is clear to one skilled in the art upon perusal of the description herein. During deployment, it is generally necessary to uncoil coil 58 to a straight conformation. In embodiments such uncoiling is performed with a deployment sleeve having a channel into which coil 60 is placed for deployment analogous to the discussed with reference to the deployment of device 10 depicted in FIG. 1. In embodiments, coil 60 is provided with an axial channel through which a deployment guide wire may be placed, analogous to the discussed with reference to deployment of device 24 depicted in FIG. 2.

Device 58 depicted in FIGS. 3A, 3B and 3C functions in accordance with the teachings of PCT patent application published as WO2006/035446 of the Applicant providing at least one stimulus that induces a perception of satiety by changing from the first conformation to the second conformation, when needed (as detected by event detector 54) to a required degree (as determined by the period of time which device 58 maintains the second conformation of coil 60).

The subject in which device 58 is deployed goes about life in the usual way. Coil 60 is ordinarily at body temperature and thus adopts the first conformation dictated by heat sensitive element 66 as depicted in FIG. 3A. In the first conformation coil 60 applies little if any pressure on duodenum 12 with only incidental contact to the duodenal walls. Due to the diameter of coil 60 which is greater than pyloric sphincter 20, coil 60 does not retreat backwards into the stomach. Coil 60 does not advance downstream in duodenum 12 as the distal portion of coil 60 is attached to control unit 30 which is fixed in place.

When the subject becomes hungry or begins to ingest food, various physiological changes occur that are automatically detected by event detector 54. The fact of detection of such an event is transmitted by event detector 54 to controller 46 in control unit 30. Controller 46 allows power from power supply unit 52 to pass through heating element 64 in coil 60 for a specified period of time as determined by timer 50. The power passing through heating element 64 heats heat sensitive element 66 to the extent that heat sensitive element 66 changes shape, forcing coil 60 to adopt the second conformation as depicted in FIG. 3B. In the second conformation, obstruction section 16 of coil 60 acts as described above to partially obstruct the lumen of duodenum 12. Further, in addition to the stimulus caused by partial obstruction of the lumen of duodenum 12 by obstruction section 16, proximal mechanoreceptor stimulating section 62 and distal mechanoreceptor stimulating section 64 apply a substantially continuous outwards pressure on the luminal walls that varies in intensity as a result of peristaltic contractions of duodenum 12, continuously inducing a perception of satiety by pressing outwards on the walls of duodenum 12, constituting mechanoreceptor-stimulating components.

After a predetermined period of time, determined with reference to timer 50 of control unit 30, controller 46 stops transferring power to heating element 64 which allows heat sensitive element 66 to cool and return to the first conformation depicted in FIG. 3A.

Coil 60 of device 58 is configured to have two conformations, a first conformation and a second conformation that provide different degrees of partial duodenal obstruction where the transformation from one conformation to another is effected with the help of a heat sensitive element 66 made of a temperature sensitive shape memory alloy. In embodiments of the present invention, a change in conformation is effected with the help of other mechanisms.

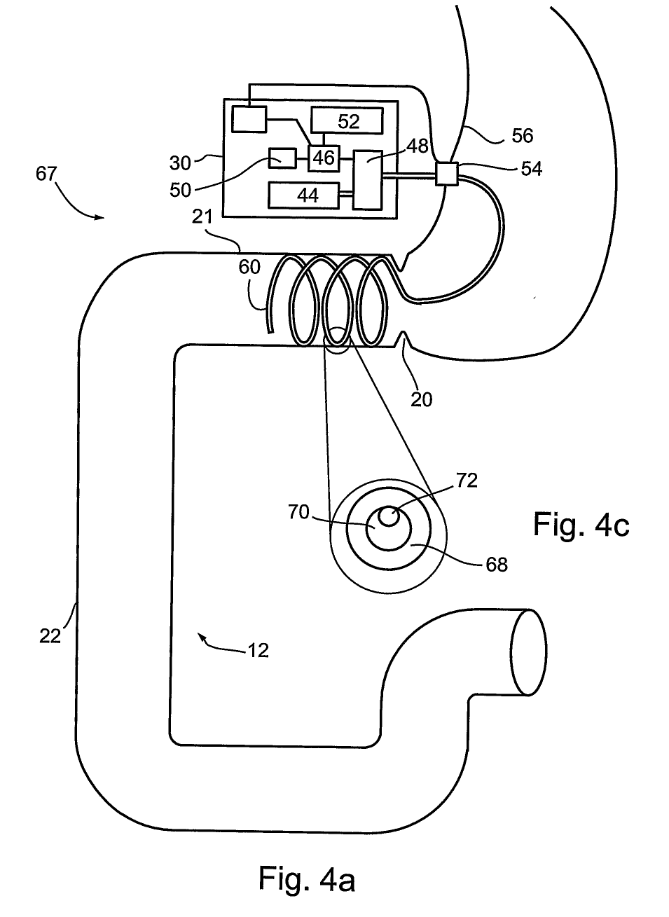

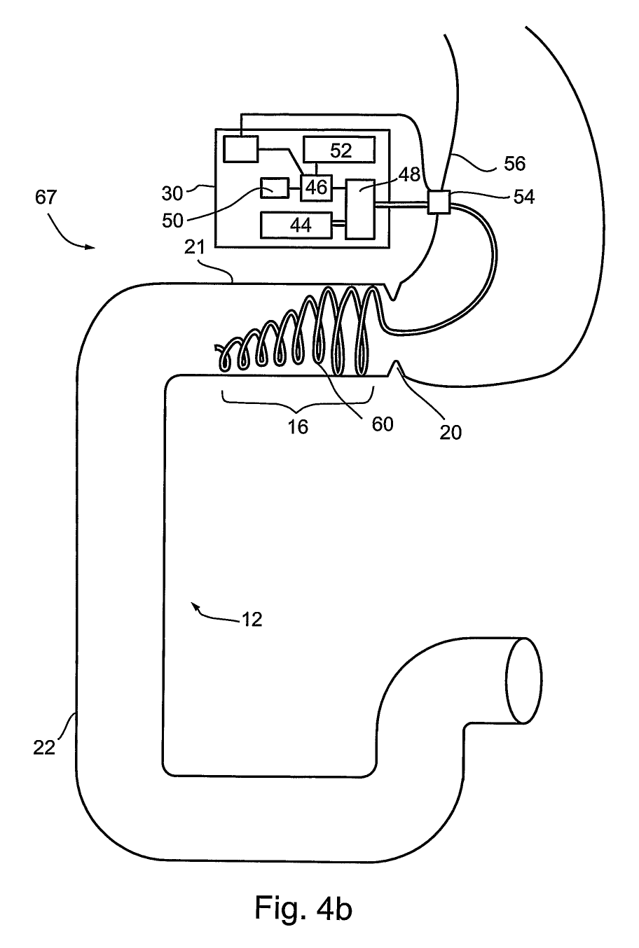

In FIGS. 4A, 4B and 4C is depicted an embodiment 67 of a device of the present invention that is substantially similar to device 58 depicted in FIGS. 3A, 3B and 3C where the change in conformation is effected by the introduction of a fluid (e.g., a gas such as air, but preferably a non-compressible fluid such as a liquid such as saline) into a closed tube.

The obstructing component of device 67 depicted in FIG. 4 comprises coil 60 which comprises a 3 mm outer diameter latex tube 68 with a 1 mm diameter channel 70 that is closed at the distal end so that channel 70 constitutes a chamber that is an elongated axial internal volume, depicted in cross-section in FIG. 4C. Passing through the length of channel 70 in coil 60 is a flexible stainless steel coiled 0.5 mm wire 72. Coiled wire 72 forces tube 68 into an approximately 3 cm long conically coiled conformation as depicted in FIG. 4B. Controller 30 includes a pressure generator 48 such as a pump or the like which is functionally associated with tube 68 so as to pump a liquid, usually contained within a fluid reservoir 44 that is a component of control unit 30, into and out of channel 70. Coiled wire 72, channel 70 and pressure generator 48 together comprise components of a mechanoreceptor-stimulating component conformation determiner and of an obstructing component conformation determiner of device 67 and function together to reversibly transform coil 60 from the first conformation to the second conformation while deployed within duodenum 12.

Tube 68, wire 72 and controller 46 together comprise a conformation-determining component of device 67 and are configured to transform the obstructing component of device 67, coil 60, from the first conformation to the second conformation while coil 60 is deployed within duodenum 12. When there is a need that coil 60 be in a first, loose, approximately 4 cm long coiled conformation depicted in FIG. 4A that obstructs duodenum 12 to a lesser degree, controller 46 activates pressure generator 48 to force fluid from reservoir 44 into channel 70 that applies a force that straightens coiled wire 72 and coil 60 in a fashion analogous to a “party whistle” having an extensile tube. When there is a need that coil 60 be in a second obstructing conformation as depicted in FIG. 4B, controller 46 activates pressure generator 48 to remove fluid from channel 70 so that tube 68 is forced to adopt the more obstructing second conformation depicted in FIG. 4B. As is clear to one skilled in the art, the change in conformation is reversed by forcing fluid back into channel 70.

An advantage of device 67 depicted in FIGS. 4A, 4B and 4C is that the degree of partial obstruction of duodenum 12 is easily varied. For example, in an embodiment such as depicted in FIGS. 4A, 4B and 4C where the transformation from the first conformation to the second conformation is in response to detection of an event of significance by event detector 54, the amount of fluid in channel 70 in the state corresponding to the second conformation can be varied. For example, if it is determined that the satiety induced by the partial obstruction of duodenum 12 by coil 60 in the second conformation depicted in FIG. 4B is too strong, the amount of fluid forced into channel 70 is increased so that the degree of partial obstruction is reduced. If it is determined that the satiety induced by the partial obstruction of duodenum 12 by coil 60 in the second conformation depicted in FIG. 4B is insufficient, the amount of fluid forced into channel 70 is decreased so that the degree of partial obstruction is increased.

In a non-depicted embodiment, a device of the present invention that is substantially similar to device 67 is provided but devoid of event detector 54. Periodically, the response of the subject to the partial obstruction of duodenum 12 by the obstructing component is evaluated, for example by a health care professional, and the degree of obstruction manually adjusted by adding or removing fluid from channel 70 to select a desired conformation similar to the first conformation depicted in FIG. 4A, the second conformation depicted in FIG. 4B, or anywhere in between.

An advantage of embodiments of the device of the present invention such as device 67 is that the outwards pressure directly applied by a coil 60 is optionally pulsatile. In such embodiments, when coil 60 has a conformation that presses against the duodenal walls (e.g., as depicted in FIG. 4B), a controller 46 activates pressure generator 48 to cyclically force fluid in and out of a channel 70, typically at a rate of between 3 Hz and 0.1 Hz. In certain instances, such pulsatile stimulation is exceptionally effective in stimulating duodenal mechanoreceptors.

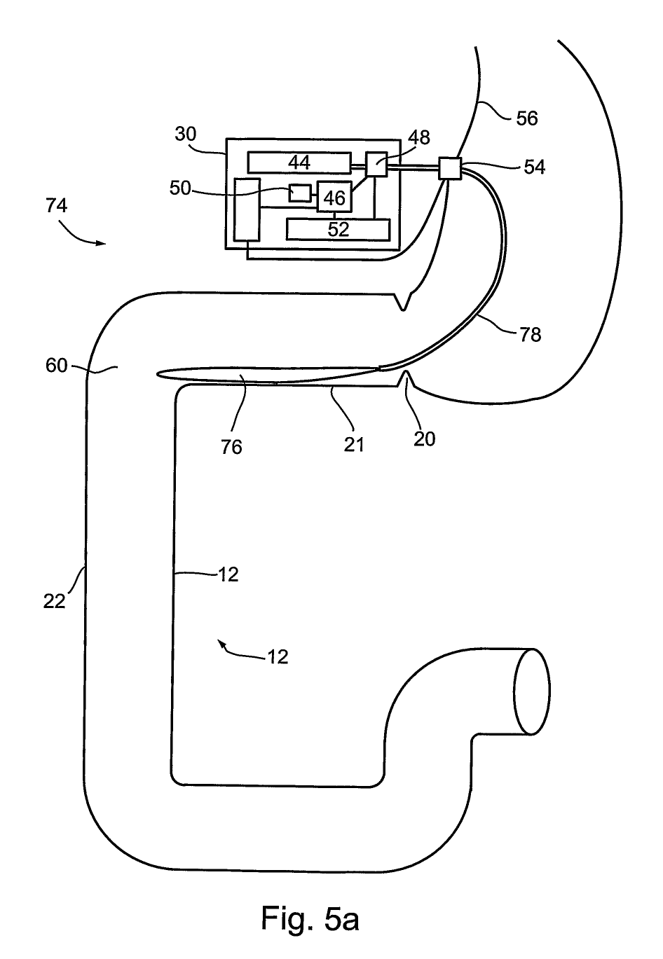

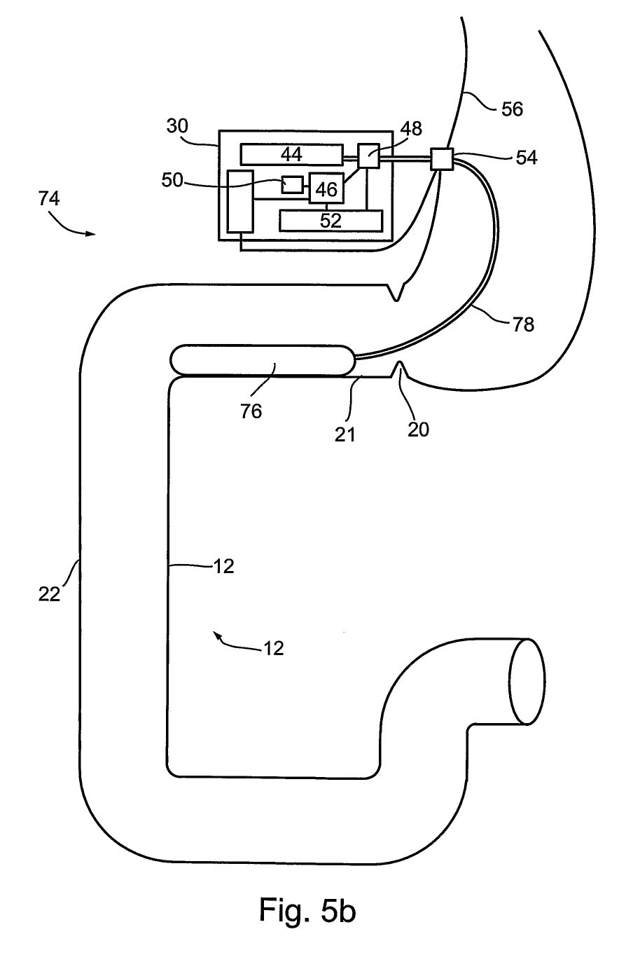

In FIGS. 5A and 5B is depicted an embodiment of the device of the present invention, device 74. Device 74 comprises a number of components including inflatable sausage-shaped balloon 76 deployed in superior portion 21 of duodenum 12 and functionally associated to control unit 30 through inflation tube 78.

Balloon 76 is a 4 cm long elongated elastic walled latex balloon with 1 mm thick walls and an inflatable volume so as to constitute an obstructing component of device 74.

Controller 30 is subcutaneously implanted in the body of a subject. Control unit 30, similarly to control unit 30 of device 67, includes a pressure generator 48 configured, upon activation by controller 46, to transport fluid (e.g., air, a liquid such as saline) from reservoir 44 through inflation tube 78 into balloon 76 so as transform balloon 76 from a first less inflated conformation depicted in FIG. 5A to a second more inflated conformation depicted in FIG. 5B. Pressure generator 48 is also configured to, upon activation by controller 48, to evacuate fluid from balloon 76, changing balloon 76 from the second conformation to the first conformation. Pressure generator 48 is a component of a conformation-determining component of device 74, being configured to reversibly transform balloon 76 from the first conformation to the second conformation while balloon 76 is deployed within duodenum 12. Controller 46 and timer 50 together comprise an actuator configured to trigger pressure generator 48 to transform the conformation of balloon 76. As noted above, depending on the embodiment such triggering may be done in response to the detection of an event of significance for changing the conformation, may be done automatically, may be done manually, and/or may be done periodically.

Inflation tube 78 is a rigid, non-elastic tube (made of materials such as polyamides (e.g., Nylon such as Nylon-12) polypropylenes, polyurethanes, polyether block amides (e.g., Pebax® available from Atofina Chemicals, Inc., Philadelphia, Pa., USA) or PEEK). Inflation tube 78 together with control unit 30 comprises an anchoring component of device 74, together maintaining the position of balloon 76 in duodenum 12. Specifically, the length of inflation tube 78 is such that the proximal end of balloon 76 commences approximately 1 cm from pyloric sphincter 20 in superior portion 21 of duodenum 12.

Deployment of a device 74 includes implanting control unit 30, deploying balloon 76 in superior portion 21 of duodenum 12 and attaching inflation tube 78 to the outlet of pressure generator 48.

The use of device 74 is substantially analogous to the described above. When in a first, not or barely inflated, conformation such as depicted in FIG. 5A, balloon 76 obstructs the lumen of duodenum 12 only to a minor degree and has substantially no effect on the subject. When in a second, more inflated, conformation such as depicted in FIG. 5B, balloon 76 partially obstructs the lumen of duodenum 12 to a greater degree.

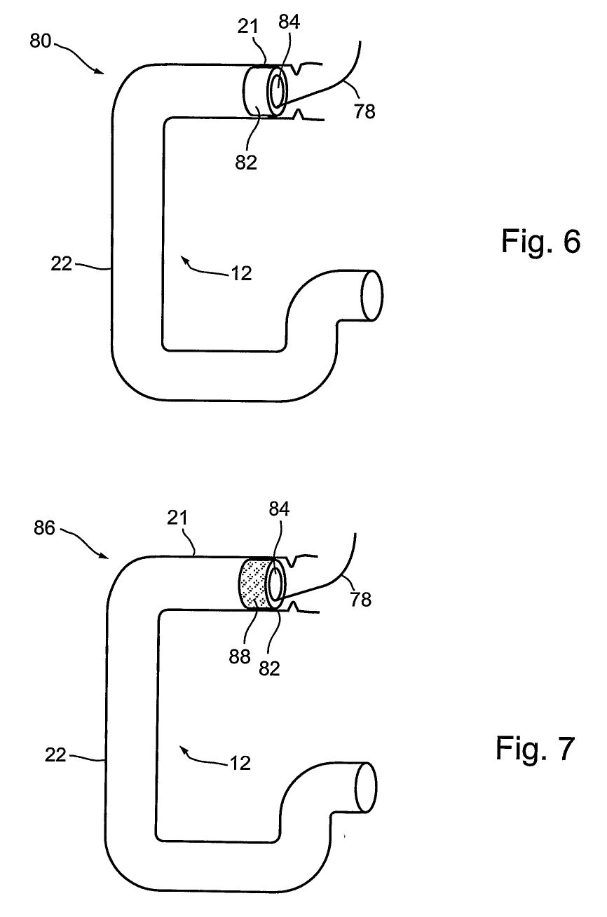

In FIG. 6 is depicted an embodiment of the device of the present invention, device 80 that is substantially similar to device 74 depicted in FIGS. 5A and 5B. Unlike device 74 which has an elongated sausage shaped balloon 76 as an obstructing component, the obstructing component of device 80 is an annular balloon 82 with a balloon lumen 84 and an external diameter. In a first, less inflated, conformation, balloon lumen 84 is relatively large and the external diameter of balloon 82 is relatively small so that balloon 82 obstructs the lumen of duodenum 12 to a lesser degree. In a second, more inflated, conformation, balloon lumen 84 constricts and becomes smaller while the external diameter of balloon 82 increases to press against the walls of duodenum 12. Thus, in the second conformation the obstruction of the lumen of duodenum 12 is greater than in the first conformation. Further, the pressure of balloon on the duodenal wall stimulates luminal mechanoreceptors of duodenum to provide a perception of satiety.

In FIG. 7 is depicted an embodiment of the device of the present invention device 86 that is substantially similar to device 80 depicted in FIG. 6. Both device 86 and device 80 comprise an annular balloon 82 as an obstructing component. However, device 86 is additionally provided with a non-expanding sleeve 88 of polyethylene terephthalate fibers surrounding the outer portion of balloon 82. In a first, less inflated, conformation, balloon lumen 84 is relatively large and the external diameter of balloon 82 is relatively small so that balloon 82 obstructs the lumen of duodenum 12 to a relatively lesser degree. In a second, more inflated, conformation, balloon lumen 84 constricts and becomes smaller while the external diameter of balloon 82 remains unchanged due to the pressure of sleeve 88. Thus, in the second conformation the obstruction of the lumen of duodenum 12 is greater than in the first conformation.

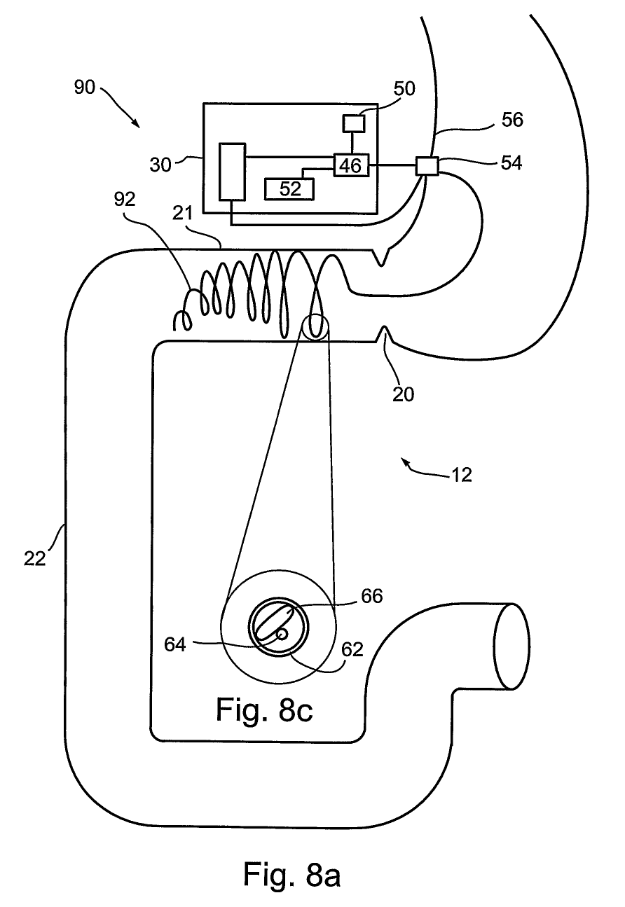

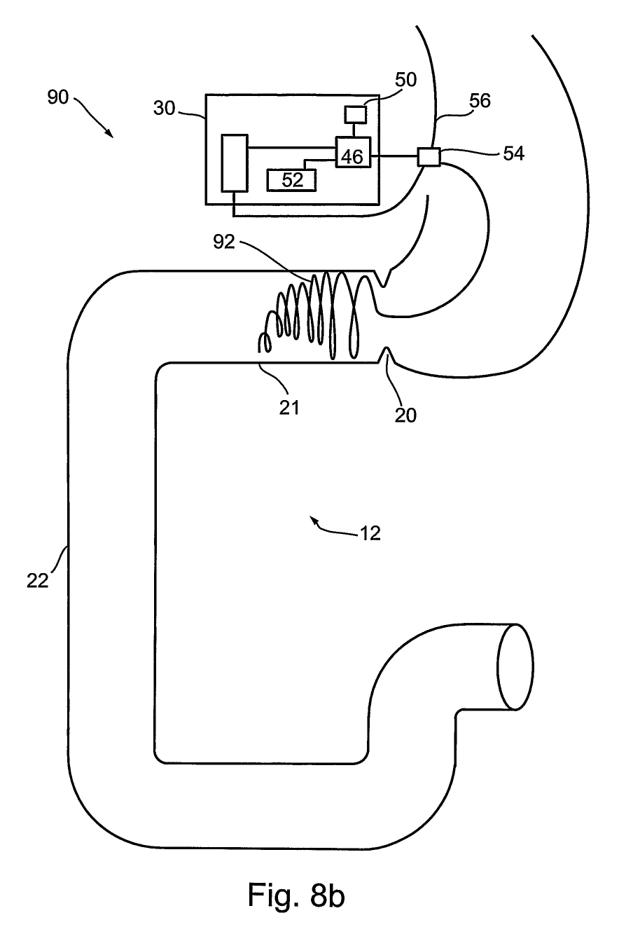

An additional embodiment of a device of the present invention, device 90, is depicted in FIGS. 8A, 8B and 8C. Device 90 is substantially similar to device 58 discussed above and depicted in FIGS. 3A, 3B and 3C and comprises some of the same components, including a duodenum stimulating coil 92 having a first lesser obstructing conformation depicted in FIG. 8A and a second greater obstructing conformation depicted in FIG. 8B. As discussed above, in duodenal stimulating coil 60 of device 58 the radii of loops making up coil 60 change to effect the transformation from a first to a second conformation. In contrast, in duodenal stimulating coil 92 the length of coil 92 is shortened from the first lesser obstructing conformation depicted in FIG. 8A to the second greater obstructing conformation depicted in FIG. 8B, by reducing the size of the gaps between the loops making up coil 92.

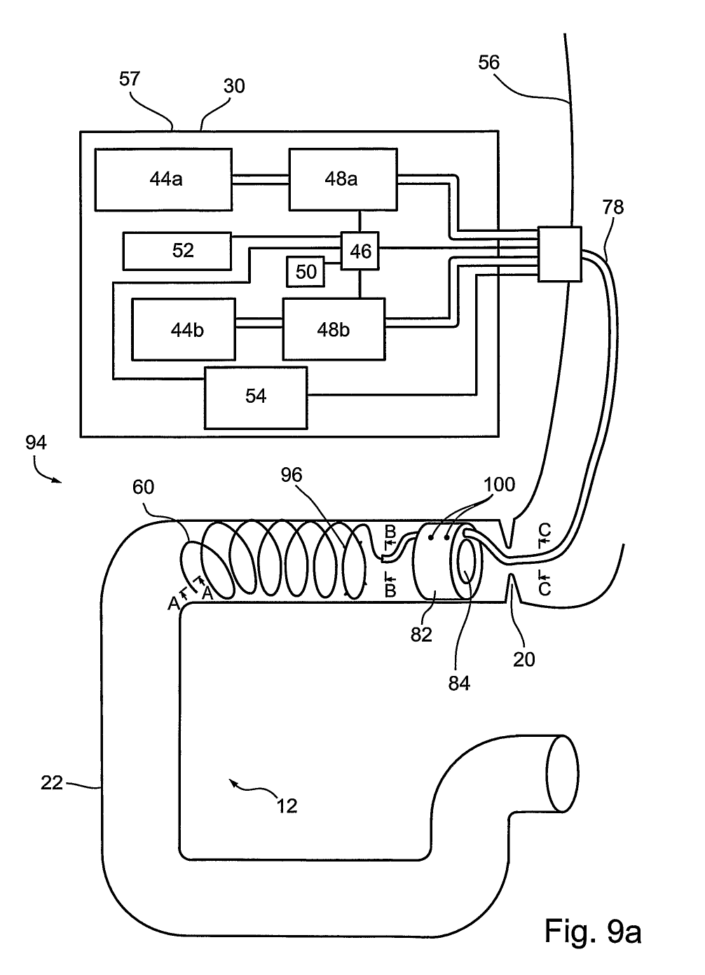

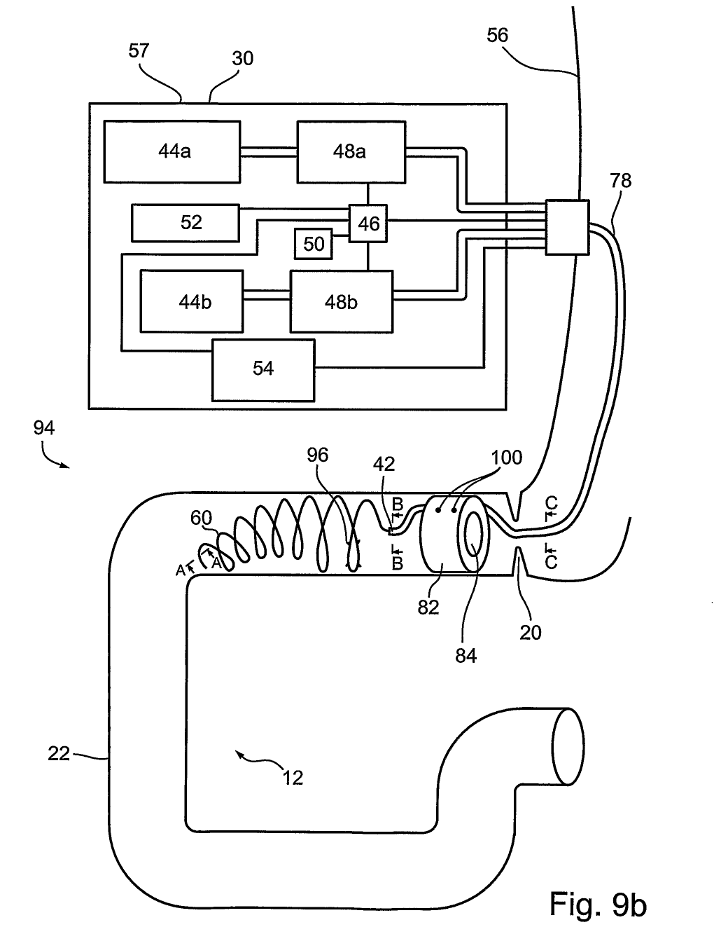

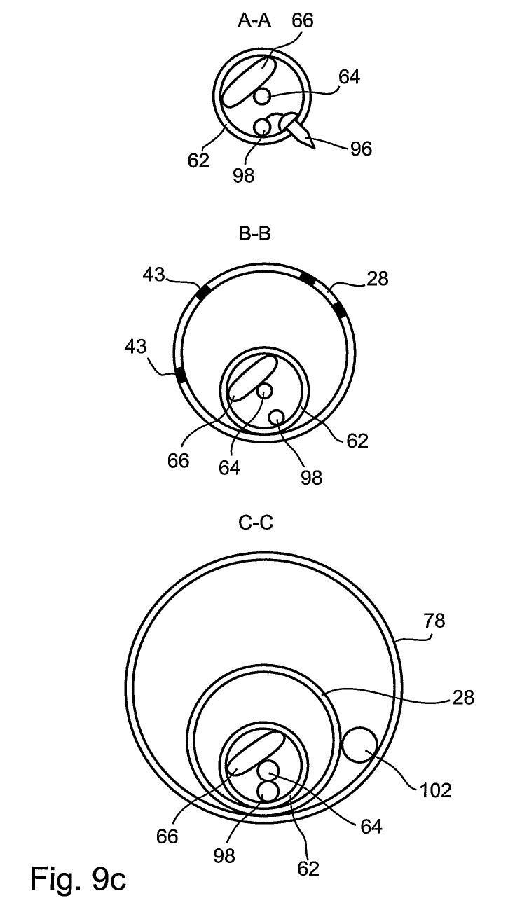

An additional embodiment of a device of the present invention, device 94, is depicted in FIG. 9A (a first lesser obstructing conformation), 9B (a second greater obstructing conformation) and 9C (cross sections across A-A, B-B and C-C of FIG. 9A). Device 94 combines features and components discussed above with reference to other embodiments of the present invention.

Similarly to device 67 depicted in FIGS. 4A-4C, device 94 comprises a first duodenum obstructing component that comprises a coil 60 having a first less obstructing conformation depicted in FIG. 9A and a second more obstructing conformation depicted in FIG. 9B. The reversible transformation of coil 60 from the first to second conformation is achieved with the help of a heating element 64 and a heat sensitive element 64 passing through tube 62 substantially as described for device 58 depicted in FIGS. 3A and 3C, see cross section A-A in FIG. 9C.

Device 94 also comprises a second duodenum obstructing component substantially similar to that of device 80 depicted in FIG. 6 that comprises an annular balloon 82 with a balloon lumen 84. As discussed for device 80, pressure generator 48b is used to force fluid from reservoir 44b into balloon 82 through inflation tube 78 or is used to withdraw fluid from balloon 82 through tube 78 into reservoir 44b, in such a way changing the conformation of balloon 82 and consequently the degree of obstruction of the lumen of duodenum 12 as well as the degree of stimulation of luminal mechanoreceptors of duodenum 12.

Device 94 also comprises an active agent dispensing component including sprayer 42 configured to administer a dose of a pharmaceutical composition comprising an active agent and a pharmaceutically acceptable carrier, substantially similar to active agent sprayer 42 of device 24 depicted in FIG. 2, cross section B-B in FIG. 9C. In device 94, a pharmaceutical composition is transported from reservoir 44a by composition pressure generator 48a through active agent feeder tube 28 and out through nozzles 43.

Device 94 also comprises four gold electrodes 96 protruding from the outwardly facing wall of coil 60 that are in electrical communication with controller 46 through leads 98 to provide electrical stimulation of the duodenum wall for vagal nerve stimulation to provide a perception of satiety as an additional mode of duodenal stimulation. The use of such electrodes to electrically stimulate the duodenum is discussed in U.S. patent application No. 60/903,289 of the Inventor, for example to electrically stimulate nerves in the duodenum. Electrodes 96 are also configured to function as physical (as opposed to only electrical) wall-stimulating features as discussed above.

Device 94 also comprises a pair of gold electrodes 100 protruding from the outwardly facing wall of balloon 82 as components of an event detector 54. Gold electrodes 100 are configured to transmit electrical signals related to electrical activity indicative of an event in the duodenal wall indicative of hunger or food ingestion. When such an event occurs, electrodes 100 transport the signals to event detector 54 through leads 102, event detector 54 then transmitting the fact of detection to controller 46.

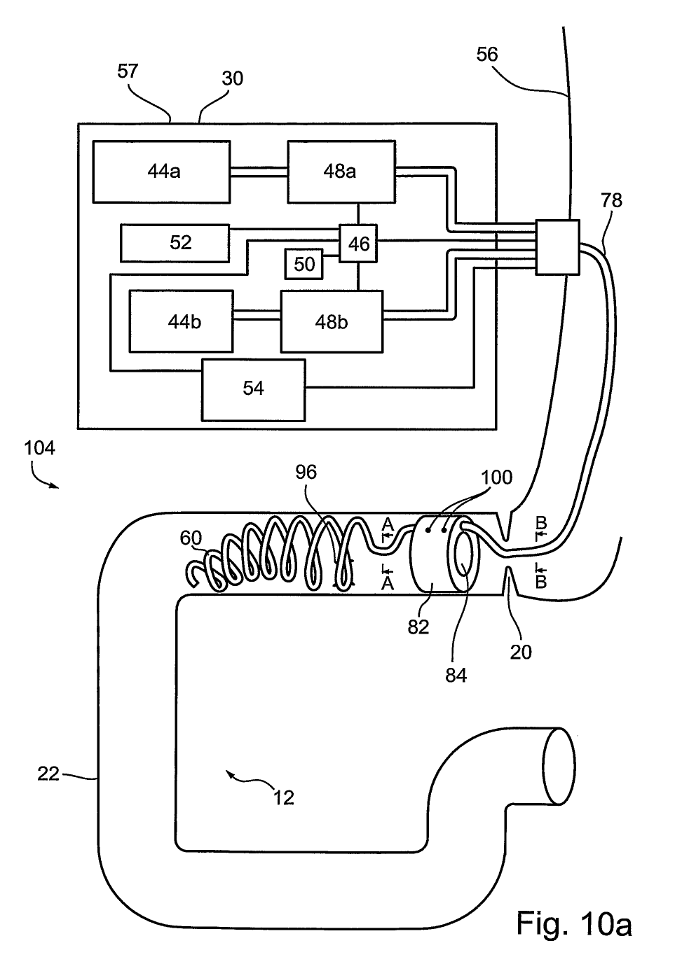

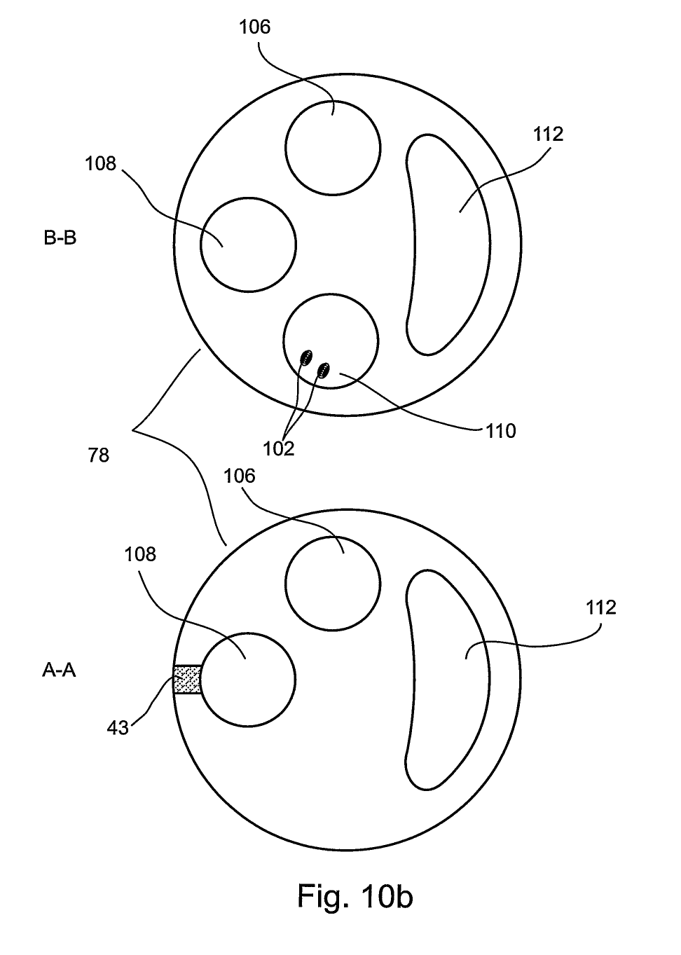

An additional embodiment of a device of the present invention, device 104, is depicted in FIGS. 10A and 10B (cross section across A-A and B-B of FIG. 10A). Device 104 combines features and components discussed above with reference to other embodiments of the present invention.

Device 104 comprises a first duodenum obstructing component that is substantially coil 60 as depicted in FIG. 10A.

Device 104 also comprises a second duodenum obstructing component substantially similar to that of device 80 depicted in FIG. 6 that comprises an annular balloon 82 with a balloon lumen 84. As discussed for device 80, pressure generator 48b is used to force fluid from reservoir 44b into balloon 82 through a lumen 106 of inflation tube 78 or is used to withdraw fluid from balloon 82 through lumen 106 of tube 78 into reservoir 44b, in such a way changing the conformation of balloon 82 and consequently the degree of obstruction of the lumen of duodenum 12 as well as the degree of stimulation of luminal mechanoreceptors of duodenum 12.

Device 104 also comprises an active agent dispensing component configured to administer a dose of a pharmaceutical composition comprising an active agent and a pharmaceutically acceptable carrier, substantially similar to the active agent sprayer of device 24 depicted in FIG. 2, cross section A-A in FIG. 10B. In device 104, a pharmaceutical composition is transported from reservoir 44a by composition pressure generator 48a through a lumen 108 in tube 78 and out through nozzles 43.

Device 104 also comprises a pair of gold electrodes 100 protruding from the outwardly facing wall of balloon 82 as components of an event detector 54. Gold electrodes 100 are configured to transmit electrical signals related to electrical activity indicative of an event in the duodenal wall indicative of hunger or food ingestion. When such an event occurs, electrodes 100 transport the signals to event detector 54 through leads 102 passing through a lumen 110, event detector 54 then transmitting the fact of detection to controller 46.

In FIG. 10B are seen cross sections of tube 78 of device 104 across A-A (distal to balloon 82) and B-B (proximal to balloon 82). Tube 78 is made of a suitable material (such as polyamides (e.g., Nylon such as Nylon-12) polypropylenes, polyurethanes, polyether block amides (e.g., Pebax® available from Atofina Chemicals, Inc., Philadelphia, Pa., USA) or PEEK) tube having a 3 mm outer diameter and includes four extruded lumina.

Lumen 106 has a 0.8 mm diameter and is displaced 0.25 mm from the edge of tube 78. As noted above, lumen 106 provides fluid communication between balloon 82 and pressure generator 48b.

Lumen 108 has a 0.8 mm diameter and is displaced 0.25 mm from the edge of tube 78. As noted above, lumen 108 provides fluid communication between composition nozzles 43 and composition pressure generator 48a, allowing administration of a pharmaceutical composition as a spray.

Lumen 110 has a 0.9 mm diameter and is displaced 0.25 mm from the edge of tube 78. As noted above, lumen 110 provides a passage for leads 102 that provide electrical communication between electrodes 100 and controller 46.

A lumen 112 has an elongated cross section with a height of 1.9 mm and a width of 0.6 mm and is configured to accommodate a strip of material, such as Nitinol, that has a coiled shape to provide coil 60 of device 104 with the shape depicted in FIG. 10A analogous to element 66 of device 58.

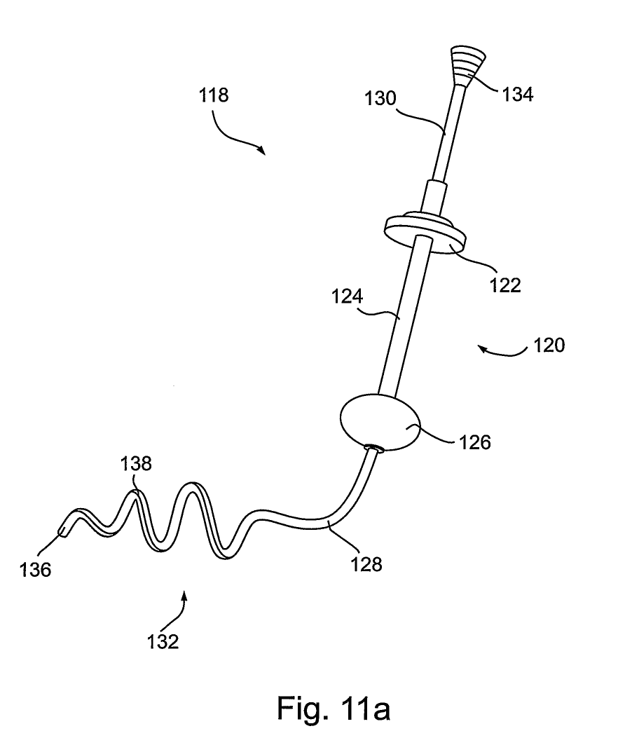

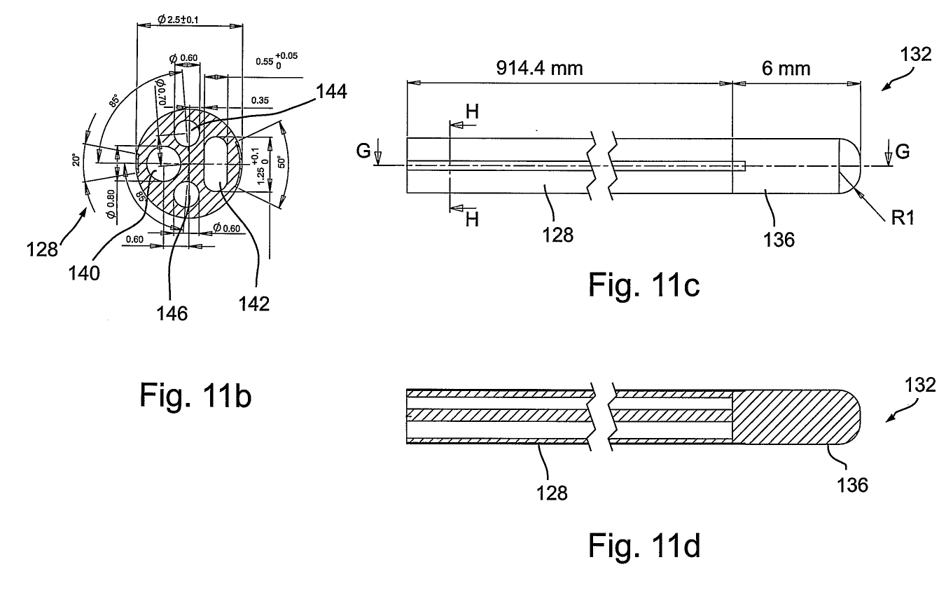

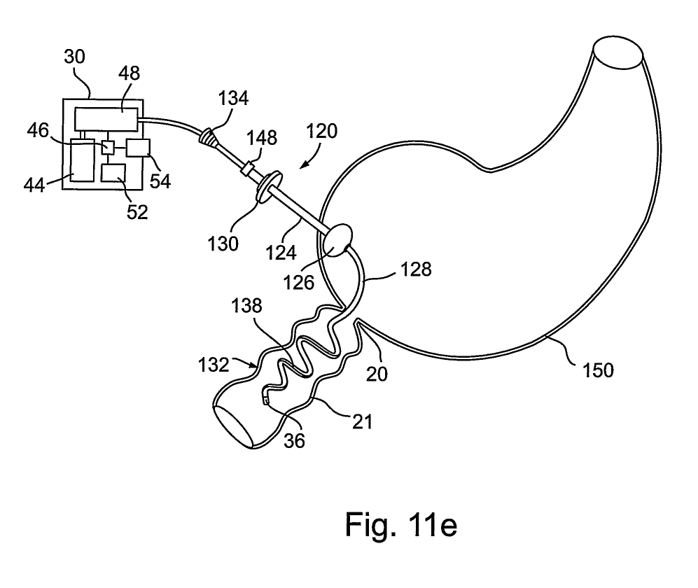

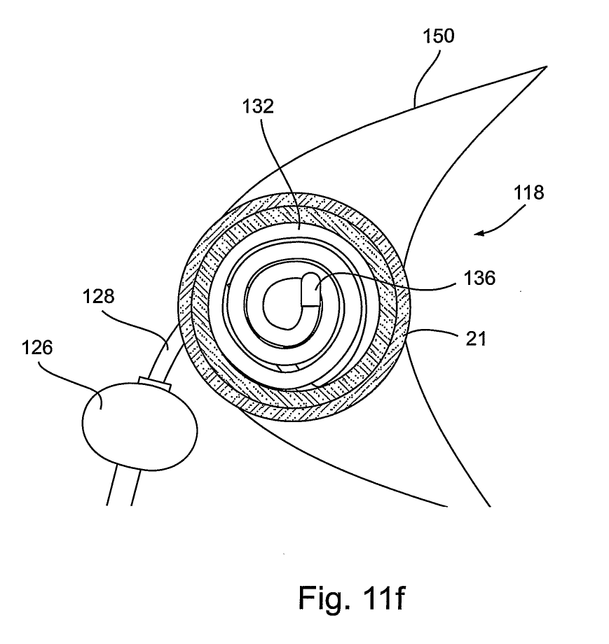

In FIGS. 11A-11F, an additional embodiment of a device including a duodenum obstructing component is depicted, device 118. In FIG. 11A, device 118 is depicted fully assembled and associated with a gastrostomy tube 120

Gastrostomy tube 120 is a standard commercially available gastrostomy tube (e.g., MicTM-“G” available from Medical Innovations Corporation, a division of Ballard Medical Products, Draper, Utah, USA) including an external button 122, a transabdominal tube 124 and an intragastric retainer balloon 126.

In FIG. 11A, it is seen that device 118 comprises a tubular body 128 having a proximal end 130 and a coiled distal end 132. Tubular body 128 has structural features so as to be configured, amongst others, as a duodenum obstructing component, an active agent feeder tube, an active agent dispensing component and as part of anchoring component to maintain the duodenum obstructing component properly positioned in the duodenum of a subject.

Proximal end 130 is provided with a connector 134 allowing connection of tubular body 128 to a pressure generator (such as a pump) and an active agent reservoir.

Distal end 132 ending with distal tip 136 has a conical coil shape so as to have an increased diameter relative to the rest of tubular body 128 and to therefore function as a duodenum obstructing component, analogously to device 10 discussed above. Similarly to previously described embodiments (e.g., device 10, device 58 or device 67), the obstructing component of device 118 coiled distal end 132 includes a flexible coiled section. As discussed above, such a coiled section effectively reduces the rate of passage of materials through the duodenum. Due to the flexibility of the coil (in device 118, both axial stretching and axial bending), solid ingested materials are not permanently caught on the obstructing component but rather, by bending and stretching of the coil, are released before any clinically significant blockage of the duodenum occurs.

Coiled distal end 132 is also configured to function as an active agent dispensing component: on the outer surface of distal end 132 is a slit 138 that functions as a spray orifice through which sprayable composition is forced out towards the luminal wall of a duodenum in which distal end 132 is deployed.

Tubular body 128 is substantially a 916 mm long by 2.5 mm diameter round flexible tube of extruded Pebax 60 resin polymerized together with 20% barium sulfate. Passing coaxially through tubular body 128 are four parallel lumens. In FIG. 11B, a radial cross-section near distal end 132 of tubular body 128, is seen the arrangement of a 0.8 mm diameter active agent lumen 140, a 0.55 mm wide by 1.25 mm high rounded-rectangle lumen 142 for accepting a Nitinol strip, and two 0.6 mm diameter round electrode guiding lumina 144 and 146.

In FIGS. 11C and 11D, axial cross sections of tubular body 128 are depicted. It is seen that a 6 mm long rounded distal cap of soft polymerized Pebax 30D is secured to tip 136 of distal end 132 of tubular body 128.

For assembly, a 914 mm long, 1 mm wide and 0.4 mm thick strip of Nitinol (not depicted) formed so that a distal end thereof adopts the desired shape of a 4 cm long conical coil having 3.5 loops is passed through rounded-rectangle lumen 142. The Nitinol strip forces distal end 132 of tubular body 128 to adopt the conical coiled shape depicted in FIG. 11A where active agent lumen 140 is on the outside of the coil. In such a way, coiled distal end 132 of tubular body 128 is configured to function as a duodenum obstructing component and also as an increased-diameter portion so as to prevent distal end 132 from moving outwards through pyloric sphincter 20 back into the stomach.

On the outer face of coiled distal end 132 of tubular body 128, a sharp knife is used to make slice 138 coaxial to tubular body 128 through the wall of tubular body 128 to active agent lumen 140.

In the art of gastrointestinal surgery, percutaneous endoscopic gastrostomy is used to endoscopically deploy a gastrostomy tube through the abdominal wall to provide a passage from the outside of the body into the stomach cavity.

In an embodiment for deploying device 118, gastrostomy tube 120 is deployed in the usual way so that external button 122 contacts the skin of a subject and intragastric retainer balloon 126 is inflated inside the stomach cavity of the subject so that bodily tissue is clamped between external button 122 and intragastric retainer balloon 126 while transabdominal tube 124 defines a direct channel from outside the body to the stomach cavity.

Device 118 is threaded through a delivery tube (e.g., 3 mm inner diameter, 4 mm outer diameter braided stainless steel flexible tube lined with polytetrafluorethylene and covered with a Pebax® polymer sleeve) forcing coiled distal end 132 into a straight conformation. While encased in the delivery tube, device 118 is threaded, distal tip 136 first through transabdominal tube 124 of gastrostomy tube 120 into the cavity of a stomach of a subject. Under guidance of and with the help of a gastroscope, distal tip 136 is guided through the pyloric sphincter and into the duodenum. The delivery tube is carefully withdrawn while device 118 is pushed forward. As distal end 132 emerges from the delivery tube, distal end 132 expands into the coiled conformation. Ultimately, distal end 132 is completely coiled and the delivery tube entirely withdrawn from gastrostomy tube 120. A retaining clip 148 is secured around tubular body 128 and against the outer side of external button 122, to act, together with tubular body 128 as an anchor so that distal end 132 is maintained in the superior portion of the duodenum.

In FIG. 11E, device 118 is depicted properly deployed in the gastrointestinal tract of a subject, where the abdominal wall and other abdominal tissue are not depicted. In FIG. 11E is seen how external button 122 contacts the skin and inflated intragastric retainer balloon 126 contacts the inner surface of stomach 150 so as to clamp bodily tissue therebetween so that transabdominal tube 124 defines a direct channel from outside the body to the cavity of a stomach 140. Connector 134 and proximal end 130 of tubular body 128 are located outside the body of the subject. Tubular body 128 passes through pyloric sphincter 20 while coiled distal end 132 of tubular body 128 is located in the superior portion of duodenum 21. Clip 148 together with the length of tubular body 128 act as an anchoring component to maintain coiled distal end 132 in the superior portion of duodenum 21 about 1 cm from pyloric sphincter 20.

In FIG. 11F coiled distal end 132 of tubular body 128, the duodenum obstructing component of device 118, is seen head-on to show the effectiveness of the partial obstruction of the lumen of the superior portion of duodenum 21 in accordance with the teachings of the present invention.

Coiled distal end 132 of tubular body 128 also functions as an active agent dispensing component. For such use, a control unit 30 is mated to connector 134. Control unit 30 includes a pressure generator 48, an active agent reservoir 44, an event detector 54 (that detects depression of a manually operable switch), a controller 46 and a power storage unit 52 and is configured to be deployed outside of the body of the subject. Control unit 30 is similar to the control unit of the DuoDopa® device (Solvay Pharmaceuticals GmbH, Hannover, Germany). When actuator 54 is triggered, controller 46 actuates pressure generator 48 to pump sprayable composition (e.g., a pharmaceutical composition including an active agent) from active agent reservoir 44, past connector 134, into active agent lumen 140. The pressure forces the sprayable composition through slit 138 as an outwardly oriented sheet-like spray, administering the composition in accordance with embodiments of the invention as discussed in U.S. patent application 60/903,289 of the Inventor. After sufficient time has passed for a desired dose to have been administered, controller 46 stops pressure generator 48 so that composition is no longer forced through slit 138 and the pressure in active agent lumen 140 is reduced. When the pressure is reduced, the elasticity of the walls of body 128 forces slit 138 closed, preventing entry of materials into active agent lumen 140.

In device 118, a prior art gastrostomy tube 120 is used to define a passage through which tubular body 128 of device 118 passes into the body of the subject.

In some embodiments, a device body such as tubular body 128 is fashioned having features (e.g., integrally formed with or attached to) of a gastrostomy tube 120 such as external button 122 and intragastric retainer balloon 126 rendering a separate transabdominal tube 124 unnecessary. In some embodiments, a different type of gastrostomy tube or functionally equivalent component is used.

In embodiments of a device of the present invention (e.g., device 24 depicted in FIG. 2, device 58 depicted in FIG. 3, device 67 depicted in FIG. 4, device 74 depicted in FIG. 5, device 80 depicted in FIG. 6 and device 80 depicted in FIG. 7) a control unit such as control unit 30 is implanted subcutaneously. Subcutaneous implantation of such objects is safe, simple and well-known in the art. That said, despite the advantages of deploying a controller of a device of the present invention subcutaneously, embodiments of the present invention include a controller deployed and/or implanted elsewhere in the body.

As noted above, embodiments of the present invention (e.g., device 24 depicted in FIG. 2, device 58 depicted in FIG. 3, device 67 depicted in FIG. 4, device 74 depicted in FIG. 5, device 80 depicted in FIG. 6 and device 80 depicted in FIG. 7) include an event detector 54 functionally associated with an actuator (e.g., comprising a controller 46) so that as a result of detection of an event of significance for changing a conformation of a duodenum obstructing component or for applying another stimulus, the event detector triggers the actuator to change the conformation of the obstructing component and/or to apply the additional stimulus. In the devices described above, event detector 54 is an electrical activity sensor configured to detect an electrical activity event in the wall of stomach 56 associated with hunger or food ingestion. One skilled in the art, upon perusal of the disclosure herein, is able to select and modify any of the different event detectors and sensors known in the art to implement of the teachings of the present invention, for example the electrode-comprising detectors disclosed in the PCT patent application published as WO 2006/035446 of the Applicant, gastric activity detectors such implemented in the Tantalus™ System (Metacure NV, MetaCure N.V., Curacao, Netherlands Antilles), or event detectors described in the U.S. patent application published as US 2005/0096637, pressure sensors (e.g., Chronicle® Medtronic, Inc., Minneapolis, Minn., USA), muscle activity sensors such as described in the U.S. patent application published as US 2004/0220633 or available from Delsys Inc. (Boston, Mass., USA), pH sensors (e.g., Bravo®, Medtronic, Inc., Minneapolis, Minn., USA).

In embodiments of the device of the present invention described above, an event detector is in wired communication with an actuator. In embodiments, an event detector is in wireless communication (e.g., radio frequency or near-infrared communication) with the actuator.

In embodiments of the device of the present invention described above, the change of conformation of an obstructing component to change the degree of duodenal obstruction or the application of another stimulus is event-driven, that is subsequently to detection of an event that is of significance for the conformation change or the application of the stimulus whether manually (by the subject or by a caregiver) or automatically (by an event detector such as event detector 54). As noted above, in embodiments the change of conformation of an obstructing component to change the degree of duodenal obstruction or the application of another stimulus is periodic and is initiated according to a periodic schedule, whether manually by the subject or care giver, or automatically with a device configured for such.

In embodiments, a device of the present invention is configured for periodic application of some duodenal stimulation by functionally associating an actuator with a timer, and the actuator is configured to periodically trigger the stimulating component with reference to the timer. In embodiments, the stimulation protocol (when, magnitude and for how long a stimulus is performed) is specified. In embodiments, the device is configured to allow the stimulus protocol to be changed or adjusted while the device is deployed in the duodenum. For example, in embodiments, a device of the present invention comprises a wireless receiver functionally associated with a controller and the controller is configured to accept commands to change the frequency or timing or other parameters of the stimulus protocol.

In the embodiments of the devices described above, pressure generator 48 comprises an electrical pump. Other suitable devices useful as pressure generators to implement the teachings of the present invention include such devices as spring-powered pressure generators, gas-pressure powered pressure generators (comprising, for example, a compressed gas reservoir and a valve) and syringes.

The embodiments of the devices described above are provided with a power supply unit comprising a power storage unit 52 (a battery). In embodiments, a power storage unit of a device of the present invention is configured to allow recharging of the power storage unit, for example as taught in U.S. patent application No. 60/903,289 of the Inventor. In embodiments, a power supply unit of a device of the present invention includes a power generation unit, e.g. a kinetic power generation unit, similar to the described in U.S. Pat. No. 6,154,422 that converts motions (such as shaking, moving or jostling) of an object with which a kinetic power generation unit is associated to electrical power.

### EXAMPLE

A study of the safety of embodiments of the teachings of the present invention was performed by deploying embodiment of a device of the present invention substantially similar to device 118 in the gastrointestinal tract of five pigs, substantially as described above with the use of a gastrostomy tube. None of the pigs suffered any apparent adverse effects from the deployment of the device, which in one case was for longer than two months.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1.-30. (canceled)

31. A device for treatment of a condition related to an eating disorder, comprising:
a) a duodenum obstructing component configured to partially obstruct the lumen of a duodenum in which deployed so as to reduce the rate of passage of materials through the duodenum; and
b) an anchoring component configured to substantially maintain a position of said obstructing component inside a duodenum wherein deployed
wherein said obstructing component comprises an inflatable balloon including an internal volume wherein said inflatable balloon is annular with a balloon lumen and an external diameter.

32. The device of claim 31, wherein said anchoring component is configured to substantially maintain a proximal end of said obstructing component no more than about 5 cm from a pyloric sphincter of a duodenum in which deployed.

33. The device of claim 31, wherein said anchoring component is configured to substantially maintain said obstructing component in the superior portion of a duodenum in which deployed.

34. The device of claim 31, said obstructing component configured to have at least two conformations, a first conformation and a second conformation providing different degrees of said partial duodenal obstruction.

35. The device of claim 34, where in said first conformation said balloon lumen is relatively large and in said second conformation said balloon lumen is relatively small, thereby providing said different degrees of said partial duodenal obstruction.

36. The device of claim 35, further comprising a conformation-determining component configured to transform said obstructing component from said first conformation to said second conformation while said obstructing component is deployed within a duodenum.

37. The device of claim 36, further comprising an actuator configure to trigger said conformation-determining component to transform said obstructing component from said first conformation to said second conformation.

38. The device of claim 31, further comprising a mechanoreceptor-stimulating component as an additional duodenum stimulating component configured to apply an outwards pressure to at least a portion of a luminal wall of a duodenum in which deployed.

39. The device of claim 38, wherein a portion of said annular balloon comprises said mechanoreceptor-stimulating component.

40. The device of claim 38, said mechanoreceptor-stimulating component configured to have at least two conformations, a first conformation and a second conformation providing different degrees of said outwards pressure.

41. The device of claim 40, where in said first conformation said balloon external diameter is relatively small and in said second conformation said balloon external diameter is relatively small, thereby providing said different degrees of said outwards pressure.

42. The device of claim 38, further comprising a mechanoreceptor-stimulating component conformation determiner configured to transform said mechanoreceptor stimulating component from said first conformation to said second conformation while said mechanoreceptor-stimulating component is deployed within a duodenum.

43. The device of claim 42, further comprising an actuator configure to trigger said mechanoreceptor-stimulating component conformation determiner to transform said obstructing component from said first conformation to said second conformation.

44. The device of claim 31, wherein said anchoring component is configured to penetrate into gastrointestinal tissue

45. The device of claim 31, wherein said anchoring component is configured to apply outwards pressure to luminal walls of a gastrointestinal tract.

46. The device of claim 31, wherein at least part of said anchoring component is configured to pass through a pyloric sphincter from a stomach associated with the duodenum.

47. The device of claim 31, further comprising a non-expanding sleeve surrounding an outer portion of said annular balloon, configured to limit the extent of outwards expansion of said annular balloon.

48. The device of claim 37, further comprising an event-detector configured to detect an event of significance for varying said degree of said partial duodenal obstruction functionally associated with said actuator.

49. A device for treatment of a condition related to an eating disorder, comprising:
a) a duodenum obstructing component configured to partially obstruct the lumen of a duodenum in which deployed so as to reduce the rate of passage of materials through the duodenum; and
b) an anchoring component configured to substantially maintain a position of said obstructing component inside a duodenum wherein deployed
said obstructing component configured to have at least two conformations, a first conformation and a second conformation providing different degrees of said partial duodenal obstruction; and
further comprising a conformation-determining component configured to transform said obstructing component from said first conformation to said second conformation while said obstructing component is deployed within a duodenum.

50. The device of claim 49, further comprising an actuator configure to trigger said conformation-determining component to transform said obstructing component from said first conformation to said second conformation.

51. The device of claim 49, further comprising an event-detector configured to detect an event of significance for varying said degree of said partial duodenal obstruction functionally associated with said actuator.

52. The device of claim 49, further comprising a mechanoreceptor-stimulating component as an additional duodenum stimulating component configured to apply an outwards pressure to at least a portion of a luminal wall of a duodenum in which deployed.

53. The device of claim 52, said mechanoreceptor-stimulating component configured to have at least two conformations, a first conformation and a second conformation providing different degrees of said outwards pressure.

54. The device of claim 49, further comprising a mechanoreceptor-stimulating component conformation determiner configured to transform said mechanoreceptor stimulating component from said first conformation to said second conformation while said mechanoreceptor-stimulating component is deployed within a duodenum.

55. A method of treatment of a condition related to an eating disorder, comprising:
a) providing a device of claim 49;
b) deploying an obstructing component of said device in the lumen of a duodenum of a subject suffering from the condition so as to partially obstruct the lumen of said duodenum; and
c) using an anchoring component of said device to substantially maintain a position of said obstructing component inside said duodenum;
thereby reducing the rate of passage of materials through said duodenum, leading to an effect beneficial for treating the condition and
further comprising:
varying the degree of said partial obstruction when necessary.

56. A device for treatment of a condition related to an eating disorder, comprising:
a) a duodenum obstructing component configured to partially obstruct the lumen of a duodenum in which deployed so as to reduce the rate of passage of materials through the duodenum; and
b) an anchoring component configured to substantially maintain a position of said obstructing component inside a duodenum wherein deployed
wherein said obstructing component comprises at least one coiled section having a conical coil shape.

57. The device of claim 56, wherein said anchoring component is configured to substantially maintain a proximal end of said obstructing component no more than about 5 cm from a pyloric sphincter of a duodenum in which deployed.

58. The device of claim 56, said obstructing component configured to have at least two conformations, a first conformation and a second conformation providing different degrees of said partial duodenal obstruction.

59. The device of claim 56, wherein a length of said obstructing component in said first conformation is different from a length of said obstructing component in said second conformation, and said degree of partial duodenal obstruction is varied by changing the length of said conical coil shaped section.

60. The device of claim 56, further comprising a conformation-determining component configured to transform said obstructing component from said first conformation to said second conformation while said obstructing component is deployed within a duodenum.
